SEQUENCE LISTING (38 sequences)

SEQ ID NO: 1 - DNA, 21 bp, artificial sequence
  misc_feature ()..(): MM211F
agataatccttgaggtccctt

SEQ ID NO: 2 - DNA, 22 bp, artificial sequence
  misc_feature ()..(): MM211R
gcccaaagtctgcctcccattc

SEQ ID NO: 3 - DNA, 22 bp, artificial sequence
  misc_feature ()..(): IT1
cgaccctggaaaagctgatgaa

SEQ ID NO: 4 - DNA, 23 bp, artificial sequence
  misc_feature ()..(): IT2
ctttggtcggtgcagcggctcct

SEQ ID NO: 5 - DNA, 24 bp, artificial sequence
  misc_feature ()..(): repetitive region (“us”)
gccttcgagtccctcaagtccttc

SEQ ID NO: 6 - DNA, 21 bp, artificial sequence
  misc_feature ()..(): repetitive region (“ds”)
cagcaacagccgccaccgccg

SEQ ID NO: 7 - DNA, 20 bp, primer
  misc_feature ()..(): ATCC-us
gattctgtgattctacaacc

SEQ ID NO: 8 - DNA, 20 bp, artificial sequence
  misc_feature ()..(): ATCC-ds
acccacagacctcttcccac

SEQ ID NO: 9 - DNA, 16 bp, artificial sequence
  misc_feature ()..(): ATCC-4
atccatccatccatcc

SEQ ID NO: 10 - DNA, 36 bp, artificial sequence
  misc_feature ()..(): ATCC-9
atccatccatccatccatccatccatccatccatcc

SEQ ID NO: 11 - DNA, 40 bp, artificial sequence
  misc_feature ()..(): ATCC-10
atccatccatccatccatccatccatccatccatccatcc

SEQ ID NO: 12 - DNA, 44 bp, artificial sequence
  misc_feature ()..(): ATCC-11
atccatccatccatccatccatccatccatccatccatccatcc

SEQ ID NO: 13 - DNA, 48 bp, artificial sequence
  misc_feature ()..(): ATCC-12
atccatccatccatccatccatccatccatccatccatccatccatcc

SEQ ID NO: 14 - DNA, 56 bp, artificial sequence
  misc_feature ()..(): competitive-us+9
gattctgtgattctacaaccatccatccatccatccatccatccatccatccatcc

SEQ ID NO: 15 - DNA, 64 bp, artificial sequence
  misc_feature ()..(): competitive-us+10
gattctgtgattctacaaccatccatccatccatccatccatccatccatccatccatccatcc

SEQ ID NO: 16 - DNA, 64 bp, artificial sequence; identical to SEQ ID NO: 15
  misc_feature ()..(): competitive-us+11

SEQ ID NO: 17 - DNA, 68 bp, artificial sequence
  misc_feature ()..(): competitve-us+12
gattctgtgattctacaaccatccatccatccatccatccatccatccatccatccatccatccatcc

SEQ ID NO: 18 - DNA, 22 bp, artificial sequence
  misc_feature ()..(): D1S191-upstream
gcatttgcttacaaatatccta

SEQ ID NO: 19 - DNA, 24 bp, artificial sequence
  misc_feature ()..(): D1S191-downstream
ctttaaaggaggactggcttgtat

SEQ ID NO: 20 - DNA, 2 bp, artificial sequence
  misc_feature ()..(): CA-1
ca

SEQ ID NO: 21 - DNA, 32 bp, artificial sequence
  misc_feature ()..(): CA-17
cacacacacacacacacacacacacacacaca

SEQ ID NO: 22 - DNA, 34 bp, artificial sequence
  misc_feature ()..(): CA-18
cacacacacacacacacacacacacacacacaca

SEQ ID NO: 23 - DNA, 36 bp, artificial sequence
  misc_feature ()..(): CA-19
cacacacacacacacacacacacacacacacacaca

SEQ ID NO: 24 - DNA, 38 bp, artificial sequence
  misc_feature ()..(): CA-20
cacacacacacacacacacacacacacacacacacaca

SEQ ID NO: 25 - DNA, 40 bp, artificial sequence
  misc_feature ()..(): CA-21
cacacacacacacacacacacacacacacacacacacaca

SEQ ID NO: 26 - DNA, 42 bp, artificial sequence
  misc_feature ()..(): CA-22
cacacacacacacacacacacacacacacacacacacacaca

SEQ ID NO: 27 - DNA, 44 bp, artificial sequence
  misc_feature ()..(): CA-23
cacacacacacacacacacacacacacacacacacacacacaca

SEQ ID NO: 28 - DNA, 46 bp, artificial sequence
  misc_feature ()..(): CA-24
cacacacacacacacacacacacacacacacacacacacacacaca

SEQ ID NO: 29 - DNA, 48 bp, artificial sequence
  misc_feature ()..(): CA-25
cacacacacacacacacacacacacacacacacacacacacacacaca

SEQ ID NO: 30 - DNA, 54 bp, artificial sequence
  misc_feature ()..(): US+CA17
atttgcttacaaatatcctacacacacacacacacacacacacacacacacaca

SEQ ID NO: 31 - DNA, 56 bp, artificial sequence
  misc_feature ()..(): US+CA18
atttgcttacaaatatcctacacacacacacacacacacacacacacacacacaca

SEQ ID NO: 32 - DNA, 58 bp, artificial sequence
  misc_feature ()..(): US+CA19
atttgcttacaaatatcctacacacacacacacacacacacacacacacacacacaca

SEQ ID NO: 33 - DNA, 60 bp, artificial sequence
  misc_feature ()..(): US+CA20
atttgcttacaaatatcctacacacacacacacacacacacacacacacacacacacaca

SEQ ID NO: 34 - DNA, 62 bp, artificial sequence
  misc_feature ()..(): US+CA21
atttgcttacaaatatcctacacacacacacacacacacacacacacacacacacacacaca

SEQ ID NO: 35 - DNA, 64 bp, artificial sequence
  misc_feature ()..(): US+CA22
atttgcttacaaatatcctacacacacacacacacacacacacacacacacacacacacacaca

SEQ ID NO: 36 - DNA, 66 bp, artificial sequence
  misc_feature ()..(): US+CA23
atttgcttacaaatatcctacacacacacacacacacacacacacacacacacacacacacacaca

SEQ ID NO: 37 - DNA, 68 bp, artificial sequence
  misc_feature ()..(): US+CA24
atttgcttacaaatatcctacacacacacacacacacacacacacacacacacacacacacacacaca

SEQ ID NO: 38 - DNA, 70 bp, artificial sequence
  misc_feature ()..(): US+CA25
atttgcttacaaatatcctacacacacacacacacacacacacacacacacacacacacacacacacaca

(19) United States
(12) Patent Application Publication
(10) Pub. No.: US 2003/0104376 A1
(43) Pub. Date: Jun. 5, 2003

(54) Assay

(76) Inventors: Veronica Brockhurst, Camira (AU); Peter Timms, Ipswich (AU); Lindsay Wolter, (US); Ross Barnard, Towong (AU); Philip Morrison Giffard, Balmoral (AU)

Correspondence Address: O'KEEFE, EGAN & PETERMAN, L.L.P., Building C, Suite 200, 1101 Capital of Texas Highway South, Austin, TX, 78746, US

(21) Appl. No.: 09/852,903

(22) Filed: May 9, 2001

Related U.S. Application Data
(60) Provisional application No. 60/202,559, filed on May 10, 2000.

(30) Foreign Application Priority Data
May 9, 2001 (WO) PCT/AU01/00526

(51) Int. Cl. C12Q 1/68

(52) U.S. Cl. 435/6

(57) ABSTRACT

The present invention relates generally to a method for identifying or otherwise detecting a nucleotide repeat region having a particular length in a nucleic acid molecule. Varying lengths of the repeat region at particular genetic locations represent nucleotide length polymorphisms. The present invention provides, therefore, a method for identifying a nucleotide length polymorphism such as associated with a particular human individual or animal or mammalian subject or for a disease condition or a predisposition for a disease condition to develop in a particular individual or subject. The method of the present invention is also useful for identifying and/or typing micro-organisms including yeasts and lower uni- and multi-cellular organisms as well as prokaryotic micro-organisms. The method of the present invention is further useful in genotyping subjects including humans. The method of the present invention is referred to herein as a “ligase-assisted spacer addition” assay or “LASA” assay.

### FIELD OF THE INVENTION

The present invention relates generally to a method for identifying or otherwise detecting a nucleotide repeat region having a particular length in a nucleic acid molecule. Varying lengths of the repeat region at particular genetic locations represent nucleotide length polymorphisms. The present invention provides, therefore, a method for identifying a nucleotide length polymorphism such as associated with a particular human individual or animal or mammalian subject or for a disease condition or a predisposition for a disease condition to develop in a particular individual or subject. The method of the present invention is also useful for identifying and/or typing micro-organisms including yeasts and lower uni- and multi-cellular organisms as well as prokaryotic micro-organisms. The method of the present invention is further useful in genotyping subjects including humans. The method of the present invention is referred to herein as a “ligase-assisted spacer addition” assay or “LASA” assay.

### BACKGROUND OF THE INVENTION

Bibliographic details of the publications referred to by author in this specification are collected at the end of the description. Reference to any prior art in this specification is not, and should not be taken as, an acknowledgment or any form of suggestion that this prior art forms part of the common general knowledge in Australia or any other country.

Microsatellites, otherwise known as Simple Sequence Repeats (SSRs), Short Tandem Repeats (STRs) or Simple Sequence Length Polymorphisms (SSLPs) consist of repetitive tracts of short DNA core sequences, of which the core units are generally between 1 to 6 base pairs. They are ubiquitous in eukaryotic genomes and are highly polymorphic due to variation in the number of repeat units they contain (Tautz, 1989). This type of length polymorphism has been estimated at being ten times more frequent than point mutations. It is this hypervariability that has seen their widespread use as DNA markers in forensics (Jeffreys et al., 1989; Gill et al., 1994), gene mapping (Hearne et al., 1992; Knowles et al., 1992), population studies addressing a wide range of questions including individual identification (Longmire et al., 1993), relatedness, parentage (Amos et al., 1993; MacDonald & Potts, 1994; Primmer et al., 1995) and intra-species comparison (Roy et al., 1994) and medical and diagnostics, dealing with a new class of neurodegenerative diseases associated with trinucleotide instability (Rousseau et al., 1991; Sutherland et al., 1993; Huntington's Collaborative Research Group, 1993; Campuzano et al., 1996).

The conventional method for detecting length variability consists of resolving polymerase chain reaction (PCR)-amplified alleles on 6% w/v denaturing polyacrylamide sequencing gels and visualization by autoradiography, either by PCR primers end-labelled with radionuclides or radioactively labelled deoxynucleotide incorporated during arplification. Several modifications have been attempted in order to simplify this procedure including alternative staining methods (Tegelstrom, 1986; Klinkicht & Tautz, 1992; Strassman et al., 1996, Vuillaume et al., 1998), modified gel matrices to increase the level of resolution (Kristensen & Dale, 1997) and the recycling of PAGE gels in order to reduce cost of reagents and time (Tereba et al., 1998).

A major problem with utilizing denaturing gel electrophoresis is that it is a time-consuming and labour-intensive technique and thus is not suitable for large throughput of samples. Multiplexing of several STR loci has been able to reduce the time required but often requires considerable optimization (Lins et al., 1996). Several techniques have recently emerged to circumvent these problems. Fluorescent gel scanning utilizing fluorescent dyes attached to PCR primers has provided an alternative detection system and has lent itself to automation, although it is still dependent on the use of polyacrylamide gels and dedicated instrumentation (Applied Biosystem 672 Genescanner System) (Taylor et al., 1994). Capillary electrophoresis allows for a more rapid separation of DNA fragments and provides resolution of units differing by as little as one base pair. However, this method is still in its infancy and is not yet amenable to automation (Mathies, 1995). Time of Flight Mass Spectroscopy (TOF-MS) has long been used to measure molecular weights. Attempts to resolve microsatellite alleles in this manner have been promising, however, resolution remains poor at sizes greater than 60 base pairs (Taranenko et al., 1999).

Another approach in the assessment of microsatellite markers has been employed by Zirvi and colleagues (1999a, 1999b). This approach utilizes a PCR/ligase detection reaction between a discriminating, labelled upstream oligonucleotide and a phosphorylated, common downstream oligonucleotide. However, the extent of misligating errors resulting from this methodology makes it far from ideal in the assessment of microsatellites.

There is thus a critical need for the development of a more efficacious technique for analyzing nucleotide repeat regions such as nucleotide length polymorphisms.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Nucleotide and amino acid sequences are referred to by a sequence identifier number (SEQ ID NO:). The SEQ ID NOs: correspond numerically to the sequence identifiers SEQ ID NO:1, SEQ ID NO:2, “etc. A sequence listing is provided after the claims.

The present invention provides a “ligase-assisted spacer addition” (LASA) assay to identify nucleotide length polymorphisms in animal including human subjects. The LASA is predicated in part on the identification of a length of spacer nucleotides capable of facilitating ligation between two nucleotide chains wherein a terminal end of one chain comprises a capturable moiety and a terminal end of the other chain comprises a moiety capable of providing an identifiable signal. The identification of a signal is indicative that the appropriate spacer has been employed. This then identifies the nucleotide length polymorphism. The method of the present invention is useful for identifying a range of disease conditions including Huntington's disease and for genotying of subjects including humans.

Accordingly, one aspect of the present invention contemplates a method for identifying or otherwise detecting a nucleotide repeat region, characterized by a particular length, in a nucleic acid molecule, said method comprising annealing to a single stranded template from said nucleic acid molecule a set of oligonucleotides, said set comprising at least two flanking oligonucleotides which are capable of annealing to nucleotide sequences on the template nucleic acid molecule flanking the nucleotide repeat region and at least one spacer oligonucleotide capable of annealing to a nucleotide sequence defining all or part of the nucleotide repeat region and wherein one of said flanking oligonucleotides is labelled with a capturable moiety and the other of said flanking oligonucleotide is labelled with a detectable moiety and subjecting the annealed molecules to a ligation reaction sufficient to permit ligation of two or more oligonucleotides if ligatably adjacent to each other and then subjecting the ligation product to conditions to facilitate attachment of the capturable moiety to a binding partner immobilized to a solid support and then subjecting the immobilized molecule to denaturing conditions to separate the template nucleic acid molecule from the annealed, potentially ligated oligonucleotides and then screening for said detectable moiety on the solid support and wherein the presence of said detectable moiety is indicative that said spacer oligonucleotide is ligated to the flanking oligonucleotides, the length of said spacer oligonucleotide thereby corresponding to the length of the nucleotide repeat region.

Another aspect of the present invention provides a method of identifying or otherwise detecting a nucleotide repeat region characterized by a particular length in a nucleic acid molecule, said method comprising annealing to a single stranded template form said nucleic acid molecule at least two flanking oligonucleotides which flank the putative nucleotide repeat region to be identified and, in a multiplicity of separate reactions, a spacer oligonucleotide of a defined length in each separate reaction which spacer oligonucleotide anneals to all or part of the nucleotide sequence between said flanking oligonucleotides wherein one of said flanking oligonucleotides is labelled with a capturable moiety and the other of said flanking oligonucleotide is labelled with a detectable moiety and subjecting said annealed molecules to ligation reactions and attachment conditions such that the oligonucleotide comprising a terminal capturable moiety anchors the annealed, potentially ligated nucleic acid molecule to a solid support; subjecting said anchored nucleic acid molecule to denaturing means such that the template nucleic strand of the nucleic acid molecule separates from the annealed oligonucleotides and then screening for said detectable moiety on a flanking oligonucleotide wherein the presence of a detectable signal is indicative that the three oligonucleotides are in tandem ligatable arrangement wherein the spacer oligonucleotide in the reaction giving the signal corresponds to the length of the nucleotide repeat region.

Yet another aspect of the present invention provides a method of identifying or otherwise detecting a nucleotide repeat region characterized by a particular length in a nucleic acid molecule, said method comprising annealing to a single stranded template form said nucleic acid molecule at least two flanking oligonucleotides which flank the putative nucleotide repeat region to be identified and, in a multiplicity of separate reactions, a spacer oligonucleotide of a defined length in each separate reaction which spacer oligonucleotide anneals to all or part of the nucleotide sequence between said flanking oligonucleotides wherein one of said flanking oligonucleotides is labelled with a capturable moiety and the other of said flanking oligonucleotide is labelled with a detectable moiety and subjecting said annealed molecules to ligation reactions and attachment conditions such that the oligonucleotide comprising a terminal capturable moiety anchors the annealed, potentially ligated nucleic acid molecule to a solid support; subjecting said anchored nucleic acid molecule to denaturing means such that the template nucleic strand of the nucleic acid molecule separates from the annealed oligonucleotides and then screening for said detectable moiety on a flanking oligonucleotide wherein the presence of a detectable signal is indicative that the three oligonucleotides are in tandem ligatable arrangement wherein the spacer oligonucleotide in the reaction giving the signal corresponds to the length of the nucleotide repeat region.

Still yet another aspect of the present invention contemplates, therefore, in a particularly preferred embodiment, a method for determining the length of a nucleotide repeat region such as in the form of a microsatellite in a target nucleic acid molecule, said method comprising the steps of:

(i) obtaining a sample of said target nucleic acid molecule;

(ii) optionally amplifying the repeat region on said target nucleic acid molecule;

(iii) subjecting the target nucleic acid molecule to denaturing conditions to yield a single stranded template carrying the repeat region;

(iv) annealing to said template three oligonucleotides separating, sequentially or simultaneously wherein two oligonucleotides are flanking oligonucleotides which are capable of annealing to the template at positions flanking the nucleotide repeat region and the third oligonucleotide is of a defined length and complementary to the nucleotide repeat region and wherein one of said flanking oligonucleotides is labelled at one end with a capturable moiety and the other flanking oligonucleotide is labelled at an end opposite to the first mentioned flanking oligonucleotide with a detectable moiety;

(v) subjecting the annealed oligonucleotides-template complex to ligation conditions such that the flanking oligonucleotides ligate to the spacer oligonucleotide if the spacer oligonucleotide is ligatably adjacent the flanking oligonucleotides;

(vi) subjecting the ligation product to anchoring conditions to capture the flanking oligonucleotide carrying the capturable moiety to a solid support;

(vii) subjecting the captured ligation product to denaturing means to release the template; and

(viii) screening for an identifiable signal wherein the presence of a signal is indicative of a spacer oligonucleotide corresponding to the length of the nucleotide repeat region.

Even yet another aspect of the present invention provides a composite nucleotide sequence comprising the structure

[x_{I}x_{II} . . . xₙ](●)ₐ[y_{I}y_{II} . . . yₘ](●)_{b}[z_{I}z_{II} . . . zₒ]

wherein

[x_{I}x_{II} . . . xₙ] and [z_{I}z_{II} . . . zₒ] are oligonucleotides of length n and o, respectively, capable of annealing to two nucleotide sequences flanking a nucleotide repeat region on a nucleic acid molecule;

(y_{I}[Y_{II} . . . yₘ] is an oligonucleotide of length m and capable of annealing to a nucleotide repeat region between the two flanking nucleotides [x_{I}x_{II} . . . xₙ] and [z_{I}z_{II} . . . zₒ];

(●)ₐ and (●)_{b} represent phosphodiester bonds between adjacent nucleotides wherein ₐ and _{b} may be the same or different and each is 0 or 1 and wherein when ₐ and/or _{b} is 0, the adjacent oligonucleotides are not ligated together;

wherein said composite oligonucleotide is formed by the process comprising annealing x, y and z separately or simultaneously to a singled stranded template nucleic acid molecule comprising a nucleotide repeat region wherein x and z anneal to regions flanking y, subjecting the molecules to ligation to generate (●)ₐ and (●)_{b} wherein ₐ and _{b} are both 1 if y is ligatably adjacent x and z on the template; immobilizing the ligated product to a solid support and subjecting the immobilized product to denaturing conditions to remove the template and then detecting the presence of the composite oligonucleotide wherein the presence of a composite oligonucleotide is indicative that y is ligatable adjacent x and z.

### BRIEF DESCRIPTION OF THE DRAWINGS

FIG. 1 is a diagrammatical representation of the Ligase-Assisted Spacer Addition (LASA) assay.

FIG. 2 is a photographic representation of a Southern blot of a 6% w/v denaturing PAGE gel containing LASA reaction products using PCR product as the template.

FIG. 3 is a photographic representation showing the analysis of 14 human samples, a negative PCR control and positive control by the LASA method and traditional denaturing polyacrylamide gel electrophoresis. Genotypes by both methods matched perfectly. No signal was generated from the negative control. Two family groups were included in the study. Group 1 consisted of mother 1 and father 1, both heterozygous 21,22, with child 1, being homozygous 21,21. Group 2, consisted of father 2, mother 2 and siblings 2.1 and 2.2, were all homozygous 20,20.

FIG. 4 is a schematic representation of the LASA methodology in the detection of Huntington's disease.

FIG. 5 is a photographic representation showing optimizations of various parameters for the extension reaction step of the LASA protocol. (A) Temperature titration (55-70° C.) was conducted using the “us” oligonucleotide with the Huntington's disease (HD)-short template. Specific products were evident at 57.6-65.7° C. (B) Higher annealing/extension temperatures were utilized for the “us27” oligonucleotide (80° C., 82° C., 85° C., 88° C., 90° C., 92° C.) and the HD-long template. Note −/+ denotes without and with Taq Polymerase in order to discriminate between the non-extended 105-mer oligonucleotide and the extended 216 bases product. (C) A titration of 0-4 mM MgCl₂ concentration provided specific HD-short extended product with the “us” oligonucleotide at 14 mM. To minimize potential interference of this component in the second ligation reaction step, the MgCl₂ concentration was subsequently maintained at 1 mM. (D) A titration of the deoxynucleotide mix (0-2 mM) containing only deoxycytosine, deoxyadenosine and deoxyguanosine, was undertaken to ensure sufficient reactants for the reaction, using “us” oligonucleotide and the HD-short template. 200 μM d(CAG)mix was deemed appropriate for this purpose. Interestingly, no product was evident at concentration higher than 500 μM. (E) Ligation times of 5, 15 and 30 minutes were compared for both the short and long extended products. Both showed the expected size growth upon ligation of the common downstream oligonucleotide (21-mer).

FIG. 6 is a photographic representation showing 20 individuals of known disease status were analysed using the LASA protocol. All were in agreement with previously characterized phenotypes.

### DETAILED DESCRIPTION OF THE PREFERRED EMBODIMENTS

The present invention is predicated in part on the development of an assay which employs varying length oligonucleotides (“spacer oligonucleotides”) to interrogate an intervening nucleotide repeat region on a nucleic acid template flanked by two oligonucleotides (“flanking oligonucleotides”) where one is labelled with a capturable moiety and the other is labelled with a detectable moiety. A spacer oligonucleotide may correspond to single or multiple repeat units. Following annealing, ligation, attachment to a solid support and denaturation to remove the template, a detectable signal is used to indicate the presence of a spacer oligonucleotide which corresponds to the length of the intervening repeat nucleotides. Only a spacer oligonucleotide corresponding to the length of the intervening sequence will be ligatably adjacent to the flanking oligonucleotide and hence capable of ligation-assisted capture of the flanking oligonucleotide with the detectable moiety via the spacer oligonucleotide to the anchored flanking oligonucleotide. The method of the present invention enables, therefore, a means for interrogating repeat nucleotide regions to identify the length of the region and the assignment of a particular polymorphism. Such a polymorphism may be associated with a particular trait, disease or a propensity to develop same, identity of a subject or identity of a particular genome. A nucleotide repeat region includes simple mono-, di-, tri- or multi-repeats or it may be complex including nested and/or non-perfect repeats. A short repeat region such as including a di-nucleotide repeat is particularly useful for genotyping of animal and human subjects. Accordingly, one aspect of the present invention contemplates a method for identifying or otherwise detecting a nucleotide repeat region, characterized by a particular length, in a nucleic acid molecule, said method comprising annealing to a single stranded template from said nucleic acid molecule a set of oligonucleotides, said set comprising at least two flanking oligonucleotides which are capable of annealing to nucleotide sequences on the template nucleic acid molecule flanking the nucleotide repeat region and at least one spacer oligonucleotide capable of annealing to a nucleotide sequence defining all or part of the nucleotide repeat region and wherein one of said flanking oligonucleotides is labelled with a capturable moiety and the other of said flanking oligonucleotide is labelled with a detectable moiety and subjecting the annealed molecules to a ligation reaction sufficient to permit ligation of two or more oligonucleotides if ligatably adjacent to each other and then subjecting the ligation product to conditions to facilitate attachment of the capturable moiety to a binding partner immobilized to a solid support and then subjecting the immobilized molecule to denaturing conditions to separate the template nucleic acid molecule from the annealed, potentially ligated oligonucleotides and then screening for said detectable moiety on the solid support and wherein the presence of said detectable moiety is indicative that said spacer oligonucleotide is ligated to the flanking oligonucleotides, the length of said spacer oligonucleotide thereby corresponding to the length of the nucleotide repeat region.

The method of the present invention is particularly useful for detecting a particular nucleotide repeat region such as defining a nucleotide length polymorphism whether or not the particular polymorphism is known. Critically, the flanking oligonucleotides flank the particular nucleotide polymorphism. The intervening nucleotide sequence is then interrogated by the varying lengths of spacer oligonucleotides. Furthermore, increased sensitivity in terms of a reduction in background is obtainable using competitive oligonucleotides designed to span the upstream flanking and repetitive regions. Competitive oligonucleotides are usefull in absorbing any stem loops formed within the template molecule. The present invention extends, in one embodiment, to the use of competitive oligonucleotides. Other factors manipulatable to decrease background signal and encompassed in a preferred aspect of the present invention include optimization of the ligation reaction and reducing the amount of template available for the LASA reaction.

Generally, but not exclusively, the method is conducted in multiple form wherein two or more spacer oligonucleotides, each of defined length, are employed. A signal is produced only when a spacer oligonucleotide is used which anneals ligatably adjacent the two flanking oligonucleotides.

Accordingly, another aspect of the present invention provides a method of identifying or otherwise detecting a nucleotide repeat region characterized by a particular length in a nucleic acid molecule, said method comprising annealing to a single stranded template form said nucleic acid molecule at least two flanking oligonucleotides which flank the putative nucleotide repeat region to be identified and, in a multiplicity of separate reactions, a spacer oligonucleotide of a defined length in each separate reaction which spacer oligonucleotide anneals to all or part of the nucleotide sequence between said flanking oligonucleotides wherein one of said flanking oligonucleotides is labelled with a capturable moiety and the other of said flanking oligonucleotide is labelled with a detectable moiety and subjecting said annealed molecules to ligation reactions and attachment conditions such that the oligonucleotide comprising a terminal capturable moiety anchors the annealed, potentially ligated nucleic acid molecule to a solid support; subjecting said anchored nucleic acid molecule to denaturing means such that the template nucleic strand of the nucleic acid molecule separates from the annealed oligonucleotides and then screening for said detectable moiety on a flanking oligonucleotide wherein the presence of a detectable signal is indicative that the three oligonucleotides are in tandem ligatable arrangement wherein the spacer oligonucleotide in the reaction giving the signal corresponds to the length of the nucleotide repeat region.

Although the present invention may be practised directly on single stranded template from a non-amplified nucleic acid molecule, in a preferred embodiment the template nucleic acid molecule is from a nucleic acid molecule which has been subjected to amplification. Any of a range of amplification reactions may be employed including PCR, rolling circle amplification and Qβ replicase based amplification amongst others.

Accordingly, another aspect of the present invention contemplates a method for identifying or otherwise detecting a nucleotide repeat region characterized by having a particular length, in a nucleic acid molecule, said method comprising amplifying a region of the nucleic acid molecule corresponding to a putative nucleotide repeat region and generating single stranded nucleic acid templates from the amplified region; annealing to the nucleic acid templates at least two flanking oligonucleotides which anneal to a nucleotide sequence flaking the putative nucleotide repeat region together with a spacer oligonucleotide of defined length capable of annealing to all or part of the nucleotide sequence flanked by said flanking primers wherein one of said oligonucleotide comprises a capturable moiety at one end and the other of said oligonucleotide comprises at an end opposite the first mentioned oligonucleotide a detectable moiety and subjecting the annealed nucleic acid-oligonucleotide complex to a ligation reaction such that two or more oligonucleotides ligatably adjacent each other are ligated together and subjecting the ligated product to an anchoring reaction to capture the capturable moiety to a solid support and then subjecting the anchored nucleic acid complex to denaturing conditions to separate the nucleic acid template away from the captured flanking oligonucleotides and then screening for the detectable moiety attached to the other of the flanking oligonucleotides wherein the presence of an identification signal indicates a spacer oligonucleotide of a length which anneals ligatably adjacent to both flanking oligonucleotides and thereby identifies the length of the nucleotide repeat region.

Reference herein to a nucleic acid molecule includes reference to double or single stranded DNA, double or single stranded RNA (including mRNA and cRNA) or a DNA/RNA hybrid. The present invention extends to cDNA as well as genomic DNA. Preferably, but not necessarily, double stranded DNA is isolated from a biological sample and subjected directly to the instant LASA method or is first subjected to amplification of a region putatively comprising a nucleotide repeat length. Alternatively, mRNA is isolated and subjected to reverse transcription such as using an RNA-dependent DNA polymerase to produce cDNA which is again either used directly in the subject LASA method or is first subjected to amplification. The biological sample is any sample putatively containing nucleic acid molecules.

In one embodiment, the biological sample is from a eukaryotic organism such as but not limited to a human, primate, livestock animal (e.g. sheep, cows, pigs, horses), laboratory test animals (e.g. mice, rats, rabbits), companion animals (e.g. dogs, cats), avian species, reptiles, fish, insects, arachnids, yeast and eukaryotic parasites such as Plasmodium species as well as plants. The eukaryotic organisms including plants may be naturally occurring, maintained in an artificial environment or be the product of genetic engineering or other genetic modification. In another embodiment, the biological sample is from a prokaryotic micro-organism. In yet another embodiment, the biological sample is a virus or viral preparation including viral nucleic acid sequences alone or integrated into a microbial or eukaryotic genome.

Most preferably, the eukaryotic organism is a human, primate or laboratory test animal or bird. Preferably, the nucleotide repeat region is a nucleotide length polymorphism such as a polymorphism associated with the presence or absence of a disease condition such as but not limited to neurodegenerative diseases including fragile X syndrome, Huntington's disease and muscular dystrophy. The method is also useful for detecting certain cancers and other malignancies. In addition, nucleotide length polymorphisms are useful in forensic science to identify a particular victim or an alleged perpetrator of a crime as well as in gene mapping and population studies. Furthermore, the method can be used to provide markers for use in identification of human and non-human individuals, plants and micro-organisms, to ascertain parentage of human and non-human animals and to monitor responses to therapies including the possibility of nucleic acid damage. The present invention further contemplates genotyping of subjects including human subjects. In this regard, the present invention extends to the identification of microsatellite markers (e.g. D1S191 which comprises [CA]ₙ repeats wherein is from about 17 to about 25.

Furthermore, the present invention provides a means of genotyping non-human animals such as mice. This is particularly important in monitoring transgenic mice and knockout mice and for use as proprietary tags. In addition, a range of murine markers have equivalent loci in other animals including humans. Examples of murine loci include but are not limited to D1Mit316, D1Mit167, D1Mit64, D1Mit167, D1Mit316, D1Mit294, D1Mit298, D1Mit428, D1Mit118, D1Mit1, D1Mit58, D1Mit160, D1Mit275, D1Mit22, D1Mit242, D1Mit168, D1Mit298, D1Mit230, D1Mit4, D1Mit231, D1Mit276, D1Mit231, D1Mit120, D1Mit432, D1Mit374, D1Mit277, D1Mit225, D1Mit410, D1Mit70, D1Mit41, D1Mit319, D1Mit374, D1Mit72, D1Mit7l, D1Mit17O, D1Mit412, Cgm3, Igf2, D1Cep4, Klk1, Pbpc2, Prkcg, Ton, Gtg3, Cds3, Fga, Fgg, Fst, Hsd3b, Prlr, Cat, D3Arb178, D3Kyol, D3Kyo2, D3Mgh16, Edn3, Il1b, Scn2a1, Sdc4, Stn1, Svp2, Ampp, Eno2, I16, Npy, Prss1, Prss2, Tac1r, Nppa, Ckb, Ighe, Calm2p3, Igf1, Myc, Prph, Acaa, Ncam, Rbp2, Thy1, Dcp1, Gh, Myh3Ppy, S1c4a1, Syb2, Ppy, Shbp, S1c4a1, Syb2, Kngt, Sst, Mdh2, Sdh, Af, Alb and Csn1. The present invention extends to homologous or equivalent loci in other animals such as humans.

The flanking oligonucleotides flank the putative nucleotide repeat region. Reference to a “oligonucleotide” is not to imply any limitation as to the size of the oligonucleotide and comprises two or more deoxyribonucleotides or ribonucleotides either naturally occurring or synthetic. The exact size of oligonucleotide may vary depending on the particular application. Preferably, the oligonucleotides range in size from about four nucleotides to about 100 and even more preferably from about eight to about 50. Oligonucleotides in the range of 10 to 30 nucleotides are particularly useful. Generally, two oligonucleotides are employed flanking the nucleotide repeat region although the present invention extends to the use of more than two oligonucleotides such as in nested primers.

The spacer oligonucleotide may range from about two nucleotides to about 400 nucleotides but is more preferably from about 2 to about 200 nucleotides and even more preferably from about 2 to about 120 nucleotides. As stated above, a set of spacer oligonucleotides are generally employed wherein each spacer oligonucleotide is of a different length. The spacer oligonucleotide is used, therefore, as a means to interrogate the length of the nucleic acid region between the two flanking oligonucleotides. Accordingly, the method of the present invention may be conducted as a single assay providing a potential “yes”/“no” answer as to the presence of a particular nucleotide length polymorphism or multiple assays may be conducted wherein a different spacer oligonucleotide is employed in separate arrays. The term “multiple” means in this context two or more assays.

One of the flanking oligonucleotides is labelled with a capturable moiety. Any number of capturable moieties may be employed such as but not limited to a biotin moiety (for binding to avidin or streptavidin), a specific nucleotide sequence interactable with a DNA or RNA binding protein, a nucleotide sequence capable of hybridizing to an immobilized primer amongst others.

Any number of detectable moieties may also be employed including those providing a fluorescent or other photonic signal, an enzyme capable of converting a substrate or a substrate convertable by an enzyme to provide an identifiable signal amongst many others. More particularly, suitable detectable molecules may be selected from a group including a chromogen, a catalyst, an enzyme, a fluorophore, a luminescent molecule, a chemiluminescent molecule, a lanthanide ion such as Europium (Eu4), a radioisotope and a direct visual label. In the case of a direct visual label, use may be made of a colloidal metallic or non-metallic particle, a dye particle, an enzyme or a substrate, an organic polymer, a latex particle, a liposome or other vesicle containing a signal producing substance and the like. A large number of enzymes suitable for use as labels is disclosed in U.S. Pat. Nos. 4,366,241, 4,843,000 and 4,849,338. Suitable enzyme labels useful in the present invention include alkaline phosphatase, horseradish peroxidase, luciferase, β-galactosidase, glucose oxidase, lysozyme, malate dehydrogenase and the like. The enzyme label may be used alone or in combination with a second enzyme which is in solution. Alternatively, a flurophore which may be used as a suitable label in accordance with the present invention includes, but is not limited to, fluorescein, rhodamine, Texas red, lucifer yellow or R-phycoerythrin.

Generally, but not exclusively, the capturable moiety is attached to the 5′ end of one primer and the detectable moiety is attached to the 3′ end of the other primer.

The solid support is preferably glass or a polymer, such as but not limited to ceramic material, nitrocellulose, polyacrylamide, nylon, polystyrene and its derivatives, cellulose and its derivatives, polyvinylidene difluoride (PVDF), methacrylate and its derivatives, polyvinyl chloride or polypropylene. A solid support may also be a hybrid such as a nitrocellulose film supported on a glass or polymer matrix. Reference to a “hybrid” includes reference to a layered arrangement of two or more glass or polymer surfaces listed above. The solid support may be in the form of a membrane or tubes, beads, discs or microplates, or any other surface suitable for conducting an assay. Binding processes to immobilize the molecules are well-known in the art and generally consist of covalently binding (e.g. cross linking) or physically adsorbing the molecules to the solid substrate.

The term “nucleotide” as used herein can refer to nucleotides present in either DNA or RNA and thus includes nucleotides which incorporate adenine, cytosine, guanine, thymine and uracil as base, the sugar moiety being deoxyribose or ribose. It will be appreciated, however, that other modified bases capable of base pairing with one of the conventional bases, adenine, cytosine, guanine, thymine and uracil may be used in the oligonucleotide primer employed in the invention. Such modified bases include, for example, inosine, 8-azaganine and hypoxanthine.

“Annealing” is used herein to denote the pairing of complementary nucleotide sequences to produce a DNA-DNA hybrid or a DNA-RNA hybrid. Complementary base sequences are those sequences that are related to the base-pairing rules. In DNA, A pairs with T and C pairs with G. In RNA, U pairs with A and C pairs with G. In this regard, the terms “match” and “mismatch” as used herein refer to the annealing potential of paired nucleotides in complementary nucleic acid strands. Matched nucleotides anneal efficiently, such as the classical A-T and G-C base pair mentioned above. Mismatches are other combinations of nucleotides which do not hybridize efficiently. The terms “hybridize” or “annealing” may, in this context, be used interchangeably.

Oligonucleotides may be selected to be “substantially complementary” to the target nucleotide sequence being tested. By “substantially complementary”, it is meant that the oligonucleotide is sufficiently complementary to hybridize with a target nucleotide sequence. Accordingly, the nucleotide sequence of the oligonucleotide need not reflect the exact complementary sequence of the target nucleotide sequence. In a preferred embodiment, the oligonucleotide contains no mismatches with the target nucleotide sequence except, in certain instances, at or adjacent the 5′ or 3′ terminal nucleotide of the target nucleotide sequence. The exact length of the oligonucleotide will depend on many factors including temperature and source of oligonucleotides and use of the method. Oligonucleotides may be prepared using any suitable method, such as, for example, the phosphodiester method as described in U.S. Pat. No. 4,356,270. Alternatively, the phosphodiester method as described in Brown et al., 1979 may be used for such preparation. Automated embodiments of the above methods may also be employed. For example, in one such automated embodiment, diethylphosphoramidites are used as starting materials and may be synthesized as described by Beaucage et al., 1981. Reference also may be made to U.S. Pat. No. 4,458,066 and 4,500,707, which refer to methods for synthesizing oligonucleotide primers on a modified solid support.

The length of the repeat region is referred to herein as a putative polymorphism. The polymorphism detectable by the present invention is also referred to herein as a “microsatellite”, “simple sequence repeat” (SSR) or “short tandem repeat” (STR). Furthermore, it covers simple or complex repeats. A complex repeat includes a nested and/or non-perfect repeat. All these terms are used interchangeably. The term “polymorphism” is also used in its broadest context and includes nucleotide length variations at a particular allele or genetic location in an individual or subject. An “individual” or “subject” may be human or non-human and covers any eukaryotic organism.

The present invention contemplates, therefore, in a particularly preferred embodiment a method for determining the length of a nucleotide repeat region such as in the form of a microsatellite in a target nucleic acid molecule, said method comprising the steps of:

(i) obtaining a sample of said target nucleic acid molecule;

(ii) optionally amplifying the repeat region on said target nucleic acid molecule;

(iii) subjecting the target nucleic acid molecule to denaturing conditions to yield a single stranded template carrying the repeat region;

(iv) annealing to said template three oligonucleotides separating, sequentially or simultaneously wherein two oligonucleotides and flanking oligonucleotides which are capable of annealing to the template at positions flanking the nucleotide repeat region and the third oligonucleotide is of a defined length and complementary to the nucleotide repeat region and wherein one of said flanking oligonucleotides is labelled at one end with a capturable moiety and the other flanking oligonucleotide is labelled at an end opposite to the first mentioned flanking oligonucleotide with a detectable moiety;

(v) subjecting the annealed oligonucleotides-template complex to ligation conditions such that the flanking oligonucleotides ligate to the spacer oligonucleotide if the spacer oligonucleotide is ligatably adjacent the flanking oligonucleotides;

(vi) subjecting the ligation product to anchoring conditions to capture the flanking oligonucleotide carrying the capturable moiety to a solid support;

(vii) subjecting the captured ligation product to denaturing means to release the template; and

(viii) screening for an identifiable signal wherein the presence of a signal is indicative of a spacer oligonucleotide corresponding to the length of the nucleotide repeat region.

Still yet another aspect of the present invention provides a composite nucleotide sequence comprising the structure

[x_{I}[x_{II} . . . xₙ](●)ₐ [y_{I}y_{II} . . . yₘ](●)_{b}[z_{I}z_{II} . . . zₒ]

wherein

[x_{I}x_{II} . . . xₙ] and [z_{I}z_{II} . . . zₒ] are oligonucleotides of length n and o, respectively, capable of annealing to two nucleotide sequences flanking a nucleotide repeat region on a nucleic acid molecule;

[y_{I}y_{II} . . . yₘ] is an oligonucleotide of length m and capable of annealing to a nucleotide repeat region between the two flanking nucleotides [x_{I}x_{II} . . . xₙ] and [z_{I}z_{II} . . . zₒ];

(●)ₐ and (●)_{b} represent phosphodiester bonds between adjacent nucleotides wherein ₐ and _{b} may be the same or different and each is 0 or 1 and wherein when ₐ and/or _{b} is 0, the adjacent oligonucleotides are not ligated together;

wherein said composite oligonucleotide is formed by the process comprising annealing x, y and z separately or simultaneously to a singled stranded template nucleic acid molecule comprising a nucleotide repeat region wherein x and z anneal to regions flanking y, subjecting the molecules to ligation to generate (●)ₐ and (●)_{b} wherein ₐ and _{b} are both 1 if y is ligatably adjacent x and z on the template; immobilizing the ligated product to a solid support and subjecting the immobilized product to denaturing conditions to remove the template and then detecting the presence of the composite oligonucleotide wherein the presence of a composite oligonucleotide is indicative that y is ligatable adjacent x and z.

In the above description, reference to “x”, ‘y’ and “z” includes reference to [x_{I}x_{II} . . . xₙ], [y_{I}y_{II} . . . yₘ] and [z_{I}z_{II} . . . z_{O}].

The present invention further contemplates the use of LASA as hereinbefore described in the manufacture of a kit for detecting and/or identifying nucleotide repeat regions such as nucleotide length polymorphisms in a eukaryotic genome.

The kit may be in any form. In one form, the kit is in compartmental form and comprises a first compartment comprising a solid support having anchored thereto a binding partner to a capturable moiety on one of at least three oligonucleotides; a second compartment adapted to contain at least three oligonucleotides, one carrying a capturable moiety at one end; another carrying a detectable moiety at an end opposite the other oligonucleotide; and a third comprising a spacer oligonucleotide; a third compartment adapted to receive a template nucleic acid molecule or a precursor form thereof; and a fifth compartment adapted to contain reagents including diluents, enzyme reagents and the like.

A single compartment kit may also be developed such as in a microtitre tray and more particularly, a multi-well microtitre tray.

The method of the present invention may also be subjected to automation to screen for a wide range of spacer oligonucleotides, such as those covering known nucleotide length polymorphisms.

Accordingly, yet another aspect of the present invention contemplates a computer program-assisted method for detecting or identifying a nucleotide length polymorphism, said method comprising:

(i) means to perform LASA or a related method; and

(ii) data processing means to record the presence of an identifiable signal and correlating same to the size of a spacer oligonucleotide.

The present invention is now described with respect to one particular preferred embodiment. This is done, however, with the understanding that the present invention extends to all variations of the subject method.

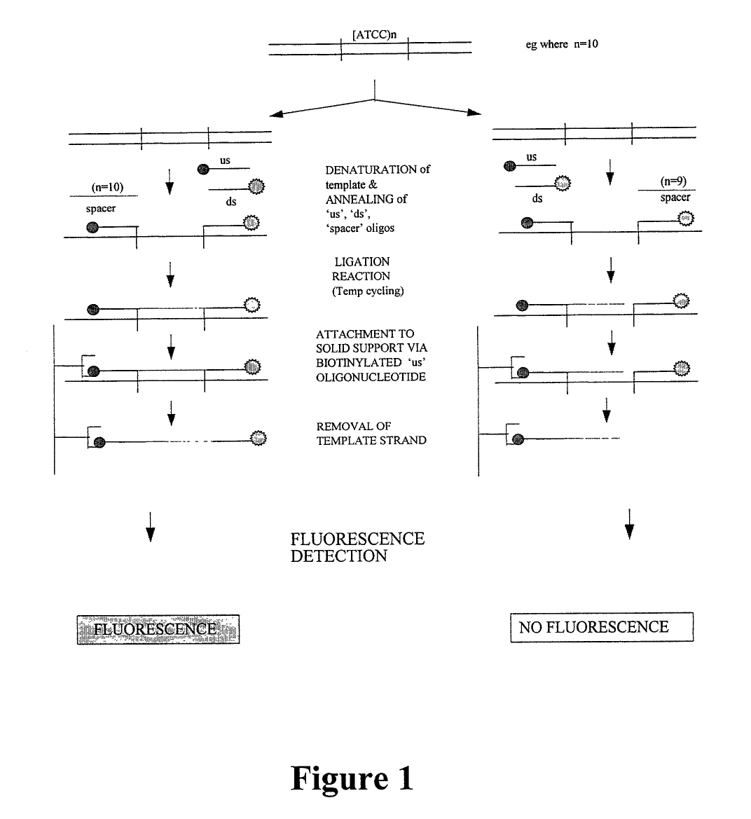

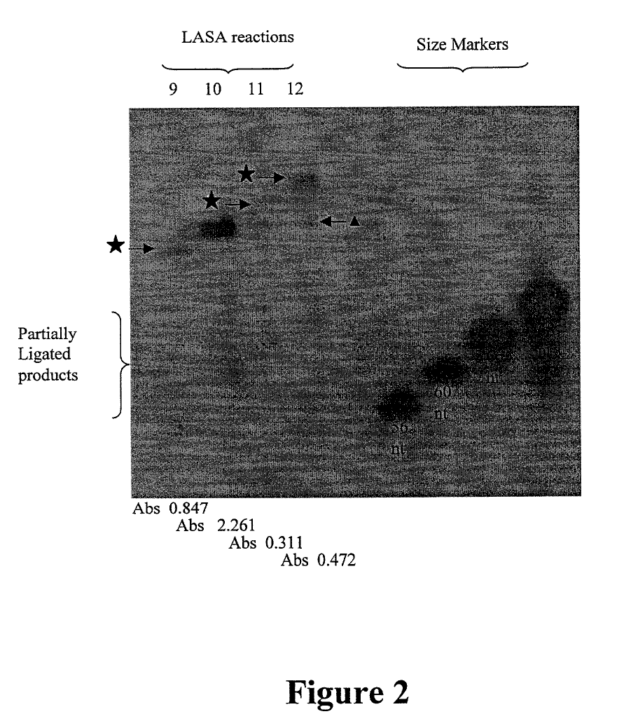

The LASA method (FIG. 1) relies on the use of three oligonucleotides. Two of the oligonucleotides (flanking oligonucleotides) flank the repeat region constituting a putative polymorphism. In FIG. 1, these two oligonucleotides are referred to as oligo “us” and oligo “ds”. Oligo “us” has a moiety at its 5′ end referred to as a “capturable moiety” which allows it to attach to a binding partner on a solid support. In one example, the capturable moiety is biotin which may be captured onto avidin or streptavidin-coated microtitre wells. Oligo “ds” has a detectable moiety such as a chromophore (e.g. fluorescein) attached at its 3′ terminus for detection purposes. A “spacer oligonucleotide” is also used as a selection or interrogation oligonucleotide which is complementary to all or part of the tandem repeat core unit. Varying lengths of the spacer oligonucleotides are added in separate reactions such that one will be of the same length as that of the repeat region being analyzed.

The LASA method requires the initial denaturation of a target nucleic acid molecule to form a single stranded template, which may also be a PCR product, spanning the microsatellite region and subsequent hybridization of the three oligonucleotides to the template. A ligation reaction follows. If the spacer oligonucleotide is ligatably adjacent both the “us” and “ds” oligonucleotides, then a single ligation product results. The ligation product is then captured onto a solid support via the oligo “us” capturable moiety and the original template is removed with, for example, alkali treatment. Successful ligation to a single ligation product resulting from the use of the appropriate sized spacer oligonucleotide is evidenced by the signal of the detectable moiety (e.g. fluorescein). The size of the spacer oligonucleotide resulting in a detectable signal then corresponds to the length of the repeat region.

As stated above, LASA may be used to detect nucleotide length polymorphisms such as occur with microsatellite repeats. Such polymorphisms are associated with a range of conditions including neurodegenerative disorders such as Huntington's disease. In a particularly useful alternative embodiment, the LASA is modified to permit extension from one of the upstream oligonucleotides with subsequent ligation in order to distinguish between short and long (CAG) expansions. This differs slightly from using spacer oligonucleotides but is still encompassed by the present invention. The modification of the instant LASA contemplated herein permits a calorimetric ELISA format or equivalent for the detriment of expanded (CAG)ₙ repeats associated with disease phenotypes including Huntington's disease. Although no intending to limit the present invention to any one particular embodiment, the modification to LASA is conveniently described in relation to Huntington's disease.

In this regard, the LASA methodology utilizes precise hybridization conditions for one of two allele-specific upstream oligonucleotides (“us” or “us27”) to preferentially anneal to its unique position on the template strand. The “us27” oligonucleotide is restricted to only hybridize to (CAG)ₙ regions with 27 or more repeats; the defined lower limit for diseased status. The ligation to a common downstream oligonucleotide, flanking the 3′ end of the (CAG)ₙ repetitive region, is facilitated by a prior extension reaction from the upstream oligonucleotide spanning the (CAG)ₙ region.

The development of this assay provides a unique means of detecting disease-related phenotypes associated with the trinucleotide repeat disorders. The ability of a modified LASA to differentiate between alleles is dependent upon (i) the correct hybridization and extension of one of the upstream oligonucleotides, and (ii) the correct ligation of the correctly extended upstream oligonucleotide. An important requirement for the extension reaction is enzyme fidelity and the absence of 5′-3′ exonuclease activity which would have otherwise displaced the downstream oligonucleotide. AmpliTaq Stoffel fragment, for example, from Applied Biosystems satisfies these requirements.

Furthermore, a concentration of 200 μM d(CAG)n optimized the assay. In addition, the extension reaction is successful over a range of MgCl₂ concentrations, of which 1 mM has minimal effects upon the ligation step. Increasing the reaction temperature provides fidelity and specificity. Two separate temperatures are deemed optimal for the different upstream oligonucleotides, being close to their melting temperature at which complete annealing could be assured Thus, the strict optimized conditions, particularly the extension temperature, are selected to promote specific binding and extension of the upstream oligonucleotides. Artefactual fragments in the extension reactions shorter than the expected products are commonly detected. However, upon ligation, these did not appear to contribute to the overall absorbance signals.

Accordingly, the present invention provides a method for discriminating between nucleotide repeat regions characterized by particular lengths in a nucleic acid molecule, said method comprising annealing to a single-stranded template from said nucleic acid molecule a set of oligonucleotides wherein one oligonucleotide anneals upstream of a putative nucleotide repeat region and is of a length which is shorter than the repeat region or is longer than said repeat region and a second oligonucleotide anneals downstream of said repeat region and wherein one of said upstream or downstream oligonucleotides is labelled with a capturable moiety and the other of said upstream of downstream oligonucleotides is labelled with a detectable moiety and then subjecting said upstream oligonucleotide to nucleotide extension conditions whereby if the upstream oligonucleotide is shorter than the repeat region, the extension product becomes ligatably adjacent the downstream oligonucleotide whereas if the upstream oligonucleotide is longer than the repeat region, then ligation is not possible with said downstream oligonucleotides such that upon ligation and immbolization to a solid support, the presence or absence of a detectable signal is indicative of an upstream oligonucleotide of a particular length and thereby a repeat region of a particular length.

Preferably, the method is conducted in duplicate with one or two upstream oligonucleotides wherein one of said oligonucleotides is potentially longer than said repeat region and the other oligonucleotide is potentially shorter than said repeat region and/or both oligonucleotides are potentially shorter than said repeat reigon.

Preferably, the method is useful for the detection of a neurodegenerative disease such as but not limited to Huntington's disease.

The present invention is further described by the following non-limiting Examples.

### EXAMPLE 1

### Microsatelite Source

The MM211 microsatellite is found within the genome of the Major Mitchell Cockatoo (Cacatua leadbeateri) and contains the tetrameric sequence ATCC. PCR conditions for amplfying an 180 base pair fragment were as follows: 1.5 mM MgCl₂, 200 μM dNTP, 100 ng genornic DNA, 0.4 mM of each primer MM211F (5′-AGATAATCCTTGAGGTCCCTT-3′) [SEQ ID NO:1] and MM211R (5′-GCCCAAAGTCTGCCTCCCATTC) [SEQ ID NO:2], 0.5 units Taq Pol Perkin-Elmer). Cycling parameters consisted of an initial denaturation at 94° C. for 5 mins, 35 cycles of 94° C. for 30 secs, 55° C. for 30 secs and 72° C. for 30 secs, with a final extension at 72° C. for 7 mins. PCR product was generated from Cockatoo sample 48404, gel-purified using Geneclean (Bio 101) and cloned into the pigment vector (Promega). The insert sequence was confirmed by automated sequencing and contained a region with 10 repeats of ATCC. This clone was used in all optimization experiments.

### EXAMPLE 2

### Specimens Containing 9, 10, 11, 12 Tetranucleotide Repeats

Sixteen Major Mitchell Cockatoo genomic DNA samples were initially genotyped using the traditional 6% w/v denaturing polyacrylamide gel electrophoresis method and detected via autoradiography. Samples were shown to contain both homozygous and heterozygous genotypes containing either 9, 10, 11 or 12 ATCC repeats.

### EXAMPLE 3

### The Ligase-Assisted Spacer Addition (LASA) Methodology

The Ligase-Assisted Spacer Addition (LASA) method (FIG. 1) relies on the use of three oligonucleotides, two of which flank the repeat region (oligo “us” and oligo “ds”). Oligo “us” has a moiety at its 5′ end to allow attachment to a solid support (such as biotin for capturing onto streptavidin-coated microtitre wells). Oligo “ds” has a chromophore attached at its 3′ terminus (such as fluorescein) for detection purposes. A “selection spacer” oligonucleotide is also used, being complementary to the tandem repeat core unit. Varying lengths of this “spacer” oligonucleotide are added in separate reactions (e.g. well 1 uses a eight-mer, well 2 uses a nine-mer, etc.) such that one will be of the same length as that of the repeat region being analyzed.

The method involves the initial denaturation of the template (CR product spanning the microsatellite region) and subsequent hybridization of the three oligonucleotides. A ligation reaction follows resulting in the joining of oligo “us” to the correct “spacer” oligonucleotide and to the oligo “ds”. The ligation product is captured onto a solid support and the original template is removed with alkali treatment. Successful ligation of all three oligonucleotides will result in the detection of fluorescence and will correspond to one spacer oligonucleotide length, thereby indicating the length of the repeat region. An optimized protocol is detailed in Table 1.

### EXAMPLE 4

### LASA conditions

Non-cycling conditions consisted of one denaturation step at 94° C. for 5 mins with a ligation step at 65° C. for 60 mins. Cycling conditions involved an initial denaturation at 94° C. for 5 mins with 99 rounds of a two-step temperature cycle of 65° C. for 1 min and 94° C. for 10 secs.

Replicate LASA reactions each underwent temperature cycling (94° C./5 mins, 99 ×(65° C./x secs, 94° C./10 secs)) with varying incubation times at the ligation step of 15, 30, 45, 60, 75 and 90 secs.

Replicate LASA reactions were set up with each undergoing temperature cycling (94° C/5 mins, 99×(65° C.160 secs, 94° C./x sees)) with varying incubation times at the denaturation step of 1, 5, 10, 20 and 30 secs.

Replicate LASA reactions were run on a Temperature Gradient PCR instrument (MJ) to ascertain the optimal ligation temperature. Temperatures were tested from 50-70° C.

LASA reactions were carried out with increasing amounts of all three oligonucleotides ranging from 0 to 100 μmoles in the reaction.

Varying quantities of PCR product (25 μl to 10-3 dilutions) was used in LASA reactions to determine the working range of template concentration for this assay. For all LASA optimizations carried out, 5 μl of PCR product was used. Concentrations of Ampligase (Epicentre Technologies) from 1-16 units were compared. LASA products were incubated in streptavidin-coated microtitre wells for between 15 and 105 minutes. Additionally, several binding buffers were compared, including 15× SSC, BW buffer (5 mM Tris pH 7.5,0.5 mM EDTA, 1 M NaCl)and PBS/0.1% w/v Tween 20.

LASA reactions were performed with 10, 20, 50, 99, 150 and 198 cycles of denaturation at 94° C./10 secs and ligation at 65° C./1 min.

### EXAMPLE 5

### Southern Blotting of Sequencing Gels

An aliquot of 1011 of each LASA reaction (9, 10, 11, 12 spacers) was loaded onto a 6% w/v denaturing polyacrylamide gel and electrophoresed at 50W at 60° C. for 1.5 hours on a Life Technology Sequencing gel apparatus prior to Southern blotting onto Hybond N⁺ membrane (Amersham) overnight using running buffer (TBE). The blot was briefly rinsed in 2× SSC and blocked for 30 minutes in 1% w/v blocking reagent (Boehringer-Mannheim) in buffer 1 (0.1 M Maleic acid, 0.15 M NaCl pH 7.5). The blot was subsequently incubated for 30 mins in a streptavidin-alkaline phosphatase conjugate (BoehringerMannheim) diluted 1:20,000 in 1% w/v blocking agent in order to detect biotinylated products. Following two 15 minute washes in 0.3% v/v Tween 20 in buffer 1, the blot was equilibrated in buffer 3 (0.1 M Tris pH 9.5, 0.1 M NaCl) and bands visualized by adding diluted CDP-Star (Boehringer-Mannheim) (1:100 in buffer 3) and x-ray exposure.

For each of the LASA reactions (9, 10, 11, 12 spacers), non-biotinylated competitive oligonucleotides were included (Table 2). The sequence of the competitive oligonucleotides were derived by combining the sequences of the upstream oligonucleotide (oligo “us”) and of a spacer oligonucleotide of length other than the biotinylated selection spacer oligonucleotide. Thus, in the “9” reaction well, a biotinylated ATCC-9 spacer oligonucleotide was added with three non-biotinylated competitive oligonucleotides; i.e. “us”+(ATCC)₁₀, “us”+(ATCC)₁₁, and “us”+(ATCC)₁₂ oligonucleotides. A titration of the competitive oligonucleotides of equi molar amounts were added to ascertain the optimal concentration in order to eliminate background signals.

### EXAMPLE 6

### Optimizadon of the LASA Parameters

In all of the optimizations, the target template used was a PCR product generated from a clone known to contain 10 repeats of the tetramer ATCC. The PCR product was thus expected to mainly contain 10 tetrameric repeats, with the possibility of other minor products due to replication slippage (stutter bands) containing greater or less than the (ATCC)₁₀ repeats.

Initially, a single step ligation reaction yielded non-specific incorporation, without discriminating between the correct and incorrect spacer oligonucleotides (Table 3). Cycling of the denaturation and ligation steps increased specificity, resulting in the signal of the correct spacer oligonucleotide being three to eight times greater (1.055) than signals from the incorrect spacer oligonucleotides (0.133-0.378).

No significant improvements were recorded by varying the time of ligation or denaturation.

Good absorbance readings were observed over the range 41-56.8° C., with a gradual decrease in readings from 56.8-72.5° C. (Table 3). At 72.5° C., no reaction products were evident, even following an overnight absorbance reading. However, it was noted that across the temperature range, background readings for the other three spacer inserts were also elevated, with a gradual decline in their intensities as they approached 65° C. To minimize background signals, all LASA reactions were subsequently performed using a ligation temperature of 65° C.

Titration of oligonucleotide concentrations between 1 to 100 pmoles showed that the LASA reaction reached a plateau at between 10 to 20 pmoles of each oligonucleotide. To maximize absorbance signals, 20 pmoles of each oligonucleotide was used in all subsequent LASA assays.

Binding of products became maximally bound after a 30 min incubation. The inclusion of 15× SSC final concentration to the reaction mix provided optimal binding conditions. Comparable signals were observed down to 0.05 μl of PCR product (equivalent to approximately 250 amoles of product). In addition, background signals decreased accordingly (Table 4).

Four units of Ampligase per reaction volume was sufficient to provide adequate reaction kinetics without compromising signal intensities.

A non-linear amplification profile was observed with increasing cycle number. At 10 cycles, absorbance signals were amplified approximately nine-fold (0.145 to 0.923). However, further increase did not result in a proportional rise in absorbance reading. A further two-fold signal amplification was achieved by altering the cycle number from 10 to 150 (Table 9).

### EXAMPLE 7

### Elimination Offalse LASA Incorporation Signals

Although the various modifications significantly improved the level of incorporation of the correct spacer insert (10×ATCC), there was a consistently elevated background level in the other spacer inserts. In particular, of the three incorrect spacer incorporations, background readings for the (ATCC)₉ spacer oligonucleotide was the highest. However, when cloned DNA (i.e. not PCR product) containing the 10 tetrameric repetitive sequence was used in the LASA reaction, background signals were still evident. This suggested that the presence of PCR stutter bands were not responsible for the background readings observed when using PCR product in the LASA assay. The inventors subsequently evaluated whether 2° structures, each as stem loop formation, may be contributing to the false incorporation.

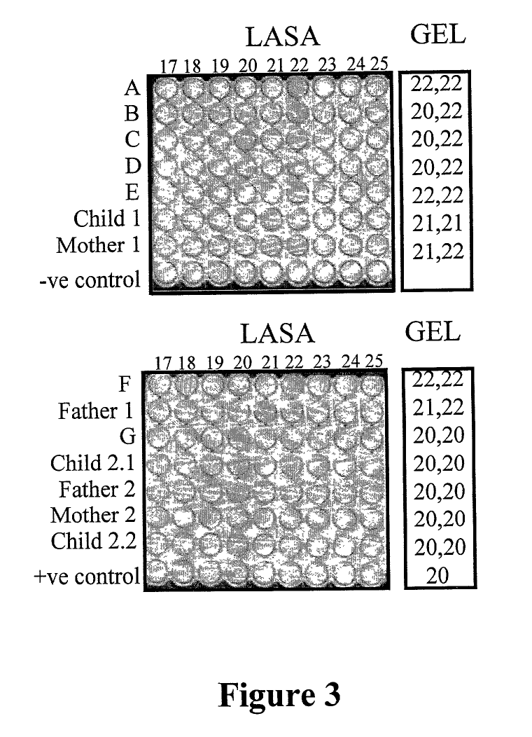

LASA products (from both PCR products and cloned DNA) were electrophoresed on a long 6% w/v denaturing PAGE gel, Southern blotted and probed for the fully ligated product. A strong signal was evident in the “10”, equivalent to a fully ligated LAS product containing 10× ATCC repeats (Abs of 2.261) (FIG. 3). The other three reactions (9, 11, 12) showed bands of lower intensities (comparable to their absorbance readings) and equivalent to lengths of 9, 11 or 12 repeats (as shown by red arrows). These same products, with similar relative intensities, were also observed when cloned DNA was used as the template for a LASA reaction. This result suggests the formation of hairpin loops within the repetitive region was thus enabling the incorporation of the incorrect spacer oligonucleotides.

However, additional bands were also noticed in the 9, 11, 12 reactions equivalent to a length of a ten-mer (shown by the blue arrow). This pattern was repeatedly observed in LASA reactions using PCR product but not when using cloned DNA, suggesting that they may be a LASA by-product from stutter artefacts.

Successful reduction of background, mainly observed in the “9” well, was achieved, however, using competitive oligonucleotides. Competitive oligonucleotides were designed to span the upstream flanking and repetitive regions. These were to act as artificial templates to hybridize to any DNA sequences formed with hairpin loops by the template. The inventors hypothesized that the correct spacer addition would be favoured and any such loop formations would be present in low concentration. A titration of competitive oligonucleotides was undertaken. Absorbance readings of LASA reactions without competitive oligonucleotides showed background level of the “9” well to be almost half of the correctly incorporated spacer oligonucleotide. Although the addition of competitive oligonucleotide reduced the overall signal of the correct spacer addition, the background level for ATCC-9 was significantly decreased; in particular, 0.2 pmoles of competitive oligonucleotide proved optimal, providing a background level drop to 15%.

A competitive LASA comparison was conducted using variable amounts of PCR product (5 μl, 1 μl, 0.1 μl) and comparing with (0.2 and 0.4 pmoles) and without competitive oligonucleotides (Table 5). Again we observed increasing background levels with increasing template concentration without the use of competitive LASA oligonucleotides. The use of 0.2 pmoles of competitive oligonucleotides, however, resulted in a drop in all background readings. Additionally, it can be seen that significant signals are demonstrated across a wide range of template concentrations. The results are shown in Tables 6 and 7.

The optimized LASA protocol is detailed in Table 1.

### EXAMPLE 8

### Use of LASA to Determine Microsatellite Length Polymorphism in Birds

Twelve bird samples were genotyped using both the conventional denaturing polyacrylamide gel electrophoresis (PAGE) and the competitive LASA assay. Scoring of PAGE and LASA alleles was done by visual inspection in both cases.

The assay was performed on unknown samples comprising of both homozygous and heterozygous genotypes. The LASA absorbance readings were able to clearly distinguish between spacer lengths, with no significant confusion by background levels (Table 10). Four heterozygous samples were apparent from the denaturing polyacrylamide gel. Samples 46766 and 28103 gave approximate 1:1 ratio between absorbance readings for the two allele lengths in the LASA method. Furthermore, sample MM100 gave signals of three-fold difference. This corresponded exactly to the band intensities on the PAGE gel.

Ten of the 12 samples had the same genotype by both methods, while two repeatedly gave discrepant results. Sample 48903 was clearly indicative of a heterozygous (9,10) by the LASA method, contrary to the homozygous (10,10) result from the denaturing gel. Additionally, sample 20970 gave high absorbance signals corresponding to three different spacer lengths (9,10,11).

### EXAMPLE 9

### Analysis of Alleles

The LASA method is modified and further optimized for the analysis of alleles associated with large numbers of repeat lengths, as in Huntington's disease. In the case of Huntington's disease, normal individuals have between 10-37 CAG repeats within the 1T15 gene. Expansions of between 37-121 repeats are observed in individuals with the disorder. The LASA method is adapted to test all of these possible variable lengths, i.e. 120-121 CAG repeats. An alternative modified system is a different solid support system thereby allowing the analysis of a large number of length possibilities. In one embodiment, a solid support (e.g. membrane) is used with covalently bound oligonucleotides (“us+spacer”) comprising the DNA sequence flanking the tandem repeat at 5′ to the tandem repeat (equivalent to the primer-us oligonucleotide) as well as a sequence containing varying numbers of repetitive units such that each of these “us+spacer” oligonucleotides vary in length by one tandem repeats, e.g. us+(CAG)₅, us+(CAG)₆, us+(CAG)₇, etc.

A mixture containing the template, ligase, buffer and a labelled oligonucleotide (“oligo-ds”) being complementary to the sequence 3′ to the tandem repeat is added onto the solid support (equivalent to primer-ds). A denaturation step and subsequent ligation step allows for the annealing of the template to the “us+spacer” oligos on the solid support as well as the oligo-ds oligonucleotide. Ligation will only occur where the “us+spacer” oligo and oligo-ds oligonucleotides hybridize perfectly to the template strand. Repeating the denaturation and ligation steps in a cycling manner using a thermostable ligase allows linear amplification of this signal. The unligated products and template stands are washed off the solid support and a simple colorimetric assay shows where ligation has successfully occurred, highlighting which tandem repeat length has been incorporated and thus detailing the number of repeat units present in the template.

### EXAMPLE 10

### Use of LASA in Human Genotyping

The D1S191 (CA)ₙ microsatellite marker has been previously characterized to contain alleles ranging from a 153 base pair allele ([CA]17 repeats) to a 169 base pair allele ([CA]₂₅ repeats) with the exclusion of the 155 base pair allele ([CA]18) in Caucasian populations (GenBank GDB 54124). LASA oligonucleotides for the DS191 LASA reactions are detailed in Table 11. Spacer oligonucleotides were designed to encompass all 9 possible lengths found within the Caucasian population. A control sample, consisting of (CA)₂₀ was cloned from a human volunteer and used to validate various assay parameters. The nucleotide content of this cloned fragment was confirmed by sequencing. PCR conditions for the D1S191 microsatellite region was described in Gyapay et al. (1994).

The ligation temperature was varied between 55° C. and ₇₀° C. and the correct incorporation of the (CA)₁₈, (CA)₁₉, (CA)₂₀, (CA)₂₁, (CA)₂₂ sized spacer oligonucleotides into the (CA)₂₀ template was evaluated. The basic LASA protocol as described in the preceding Examples was followed.

The effect of including competitive oligonucleotides (non-biotinylated oligonucleotides comprising of the upstream flanking and repetitive regions (Table 11) on the assay performance was evaluated by repeating the titration detailed above (55-70° C.), with the inclusion of 0.2 pmoles of each competitive oligonucleotide.

The effect of varying NaCl concentrations (0 to 0.9 M) during a separate initial denaturation phase was assessed by adding various NaCl concentrations to 1 μl of PCR product from the cloned standard in a final volume of 11 μL The mixture was denatured prior to the addition of the LASA oligonucleotides and ligation reagents, in contrast to the standard assay procedure in which one reaction mixture containing all of the components were initially denatured for 5 minutes and subsequently cycled between 65° C. and 94° C. for denaturation/ligation. Thus, in this modification, the sample mix was incubated at 94° C. for S minutes and allowed to cool at 4° C. for 10 minutes using a Hybaid PCR instrument. Subsequent addition of ampligase buffer (Epicentre Technologies), 20 pmoles of each spacer oligonucleotide, upstream and downstream oligonucleotides and 4 units of Ampligase (Epicentre Technologies) in a final volume of 20 μl were carried out.

Conditions as described above were replicated using only one NaCl concentration (0.18 M) with a temperature titration from 55-70° C. on a Hybaid PCR Express instrument.

In an attempt to reduce the production of stutter bands during PCR amplification, the number of PCR cycles was reduced from 35 to 30. Taq Polymerase (Perkin-Elmer) was replaced with Platinum Taq Polymerase (Roche).

Fourteen human samples, including 2 family groups, were involved in this study. 5 μl of each human sample PCR product was utilized in 9 separate reactions using each of the respective spacer oligonucleotides. Only 1 μl of cloned standard PCR product was used in this assay. Colour was allowed to develop overnight and measured in a UV plate reader at 405 nm.

Biotinylated PCR products from each sample were fractionated on a 6% v/v urea polyacrylamide gel using a Life Technology sequencing gel electrophoresis apparatus at 50W for 2.5 hours (60° C.). Southern blotting of the denaturing gel onto Hybond N+(Amersham) using 1×TBE as the transfer medium and detection of biotinylated products using streptavidin-alkaline phosphatase conjugate (Roche) (1/100 dilution) revealed fragment lengths.

Using the non-competitive format, there was relatively little discrimination between the correctly and incorrectly matched spacer oligonucleotides across the range of ligation temperatures from 55-70° C. (Table 12). Although the signal to noise ratios in the current dinucleotide LASA assay were slightly reduced with the added competitive oligonucleotides (Table 12), it was insufficient to provide clear differentiation between the correct (CA)₂₀ spacer oligonucleotide and the incorrectly matched spacer oligonucleotides. A titration of the competitive oligonucleotide concentration from 0 to 10 pmoles did not improve the LASA signal to noise ratios. A ligation temperature of 65° C. was selected for ligation in agreement with the reported optimal temperature for enzyme efficiency (65° C.) (Epicentre Technologies).

Denaturation of the template in the presence of NaCl ions prior to the two-step ligation/denaturation cycling reaction, did enable good discrimination between the correct and incorrect spacer oligonucleotides (Table 13). A concentration of 0.18 M NaCl was optimal with background signals reduced to less than 22% of the correctly matched spacer oligonucleotide. This effect was non-existent at higher salt concentrations (>0.36 M) and demonstrated a dramatic reduction in overall absorbance readings, probably due to the inhibition of the ligation reaction kinetics by the salt.

Using an initial prior denaturation of the template in 0.18 M NaCl, a temperature titration (55-70° C.) showed that signal to noise ratios were improved across all temperatures (Table 14). A temperature of 65.7-67.4° C. produced the lowest signal to noise ratios, without compromising the overall absorbance signal of the correctly matched spacer oligonucleotide. The inventors selected 66° C. as the optimal ligation temperature for the D1S191 dinucleotide LASA assay.

From each of the 14 human subjects, PCR product was separated on a 6% w/v denaturing polyacrylamide gel to ascertain allele lengths. Stutter bands were prevalent in all lanes, including the cloned standard. The inventors reduced the level of stutter bands by decreasing the amplification cycle number from 35 to 30 and by using an alternative polymerase source (Platinum Taq Polymerase-Roche).

The D1S191 allele was easily scored via the LASA assay for all 14 human samples (FIG. 3). When compared to the allele sizes as determined by the traditional polyacrylamide gel electrophoresis, results matched perfectly. The negative control did not show any contamination problems, although strict guidelines were followed during the set up of these reactions in order to minimize such an occurrence.

Two family groups were included in this study. Family group 1 consisted of parents, both heterozygous (21,22), and a child with an homozygous (21,21) genotype. Family group 2, comprising of two parents and two siblings, were all homozygous (20,20) genotypes. This is in agreement with traditional Mendelian inheritance laws.

The success of the LASA method for the detection of dinucleotide length polymorphisms relied upon (i) the modification of the LASA reaction to include an initial denaturation of the template in the presence of 0.1S M NaCl prior to the addition of the LASA reagents, and (ii) the reduction of PCR stutter bands. The modified LASA assay correctly determined the D1S191 genotype of 14 human samples, offering significant advantages over gel-based methods. This ELISA-based methodology is able to overcome the drawbacks presented by the cumbersome gel-based protocol by being less technically-demanding, more time efficient, cost effective and amenable to automation.

### EXAMPLE 11

### Use of LASA in the detection of Huntington 's disease

Polyglutamine expansions within the IT15 gene on chromosome 4 gives rise to Huntington's disease (HD Collaborative Research Group, 1993), having a prevalence of I in 10,000 in the Caucasian population. This disorder manifests itself during mid-life and is characterized by involuntary body movements, intellectual and psychological decline. Physical and psychological symptoms progressively worsen, incapacitating the individual over a 10-20 year period, eventually leading to death. Huntington's disease is devoid of medical treatment except for pharmacological therapy to aid with presenting symptoms.

This Example describes the use of LASA to detect Huntington's disease, avoiding both gel electrophoresis and Southern transfer analysis. The Allele-Specific Extension and Ligation (A-SEaL) methodology (also known as LASA) relies upon the selective and specific hybridisation of one of two allele-specific upstream oligonucleotides. The LASA is employed such that the ligation of the upstream oligonucleotide to a downstream oligonucleotide is dependent upon polymerisation across the (CAG) region that is filled by deoxynucleotides via an extension reaction. Using this technique, the inventors were able to differentiate individuals into two categories—those possessing normal or expanded alleles.

Parameters for amplifying a region containing the (CAG)ₙ repeats within the IT15 gene were as follows: 1 mM MgSO₄, 200 μM dNTPs, 200 ng genomic DNA, 2×PCR Enhancer reagent (Roche), 10 pmoles of each primer, IT1 (5′-CGACCCTGGAAAAGCTGATGAA-3′ [SEQ ID NO:3]) and IT2 (5′-CTTTGGTCGGTGCAGCGGCTCCT-3′ [SEQ ID NO:4]), 1×PCR Enhancer buffer and 0.5 units of Taq polymerase (Perkin-Elmer). Cycling conditions consisted of an initial denaturation at 94° C. for 5 mins, 30 cycles of 94° C. for 30 secs, 59.5° C. for 1.5 mins, 72° C. for 1 min, with a final extension step at 72° C. for 5 mins.

PCR product was generated from sample HT10 and cloned into PCR-script (Stratagene). Two clones were produced—“Huntington's disease(HD)-short” contained (CAG)₂₀ and “BD-long” contained (CAG)₁₄. Inserts were confirmed by sequencing. PCR products of these clones were used in all optimization experiments.

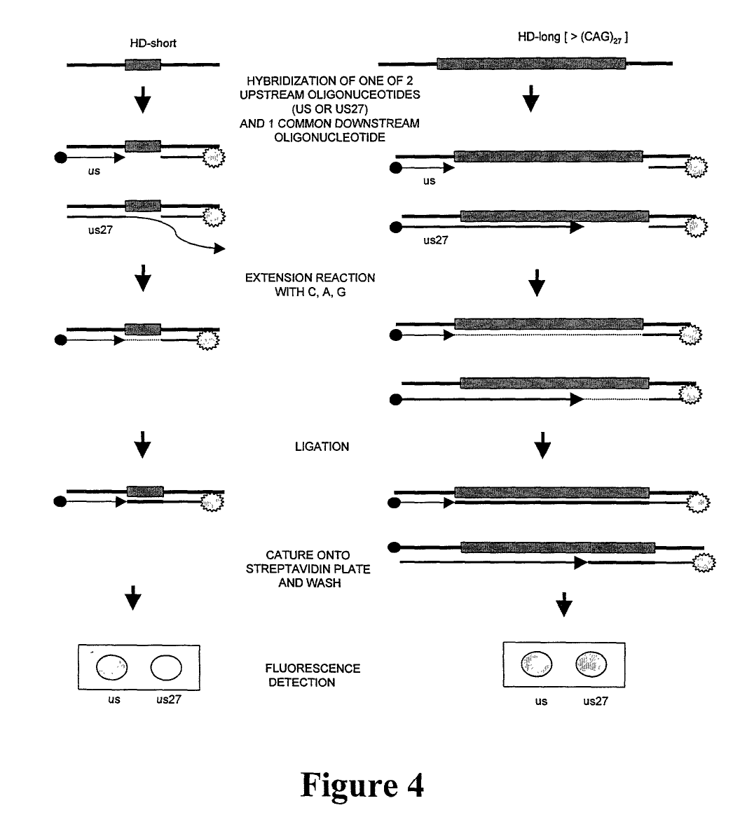

The LASA methodology was performed as described above. Two separate reactions were required, each containing one allele-specific interrogative upstream oligonucleotide (“us” or “us27”) and one common downstream oligonucleotide (“ds”). The “us” oligonucleotide represents the base sequence immediately upstream of the (CAG)ₙ repetitive region (“us”: 5′-GCCTTCGAGTCCCTCAAGTCCTTC-3′ [SEQ ID NO:5]), whilst the “us27” oligonucleotide (105-mer) is equivalent to the “us” sequence with an additional (CAG)₂₇ included in the sequence. The upstream oligonucleotides also have a biotin moiety at their 5′ end for capture onto streptavidin-coated microtitre wells. The downstream oligonucleotide (“ds”) represents a base sequence immediately following the (CAG)ₙ repetitive region (5′-Phosphorylated CAGCAACAGCCGCCACCGCCG-3′ [SEQ D NO:6]) and has a fluorescein label attached at its 3′ terminus for detection purposes. As described above, the method relies upon the specific and differential hybridization of the upstream oligonucleotides (“us” or “us27”). Subsequent extension using deoxynucleotides and a final ligation step will identify whether either of the two upstream oligonucleotides were successful in annealing specifically to the template strand. A non-displacing enzyme is used to prevent unwanted extension products under conditions in which only those deoxynucleotides predicted by the template are supplied in the reaction, in this case, deoxycytosine, doexyadenosine and deoxyguanosine. Resulting products are captured onto streptavidin-coated microtitre wells and visualized colorimetrically (FIG. 4). The assay is based on the assumption that the “us27” oligonucleotide should only anneal to targets containing (CAG)₂₇ repeats or more. Since trinucleotide CAG expansions less than 26 units have never been associated with Huntington's disease, any repetitive region greater than (CAG)₂₆ is suspect of the disorder. The “us” oligonucleotide is designed to act as a positive control and should be positive for all reactions. An optimized protocol is shown in Table 15.

In order to maximize the amount of extended product, whilst the potential effects of these reaction components upon the secondary ligation step, the inventor focussed primarily on the first step, the extension reaction. Linear amplification of extension products was achieved by thermal cycling, involving an initial denaturation at 99° C. for 5 mins with subsequent cycling between annealing and denaturation temperatures.

Both HD-short and HD-long PCR products were tested using “US” and “us27” oligonucleotides respectively. 10 μl reactions containing 1 mM MgCl₂, 200 uM d(CAG) mix, 1 μl cloned standard PCR product, 20 pmoles of upstream and 20 pmoles of downstream oligonucleotides and 0.5 units AmpliTaq Stoffel fragment (Applied Biosystems) were prepared. Temperature cycling on a Hybaid PCR Express instrument for the “us” and “us27” oligonucleotides were as follows: 94° C. for 5 mins, (94° C. for 10 secs, 55-70° C. for 10 secs)×99. A secondary titration for the “us27” oligonucleotide was conducted. Cycling for the ′us27” oligonucleotide was 94° C. for 5 mins, (94° C. for 10 secs, 80° C./82° C./85° C./88° C./90° C./92° C. for 10 secs)×99.

Reactions containing 0 to 4 mM MgCl₂, 200 μM d(CAG) mix, 20 pmoles of each oligonucleotide, 1 μl of HD-short PCR product and 0.5 units AmpliTaq Stoffel fragment were undertaken. Cycling was as follows: 94° C. for 5 mins, (94° C. for 10 secs, 65° C. for 10 secs)×99.

Reactions containing 0-2 mM d(CAG) mix, 1 mM MgCl₂, 20 pmoles of the “us” and “ds” oligonucleotides and 0.5 units AmpliTaq were conducted and subjected to thermal cycling of 94° C. for 5 mins, (94° C. for 10 secs, 65° C. for 10 secs)×99.

1 unit of T4 DNA ligase (Roche) and appropriate buffer was added to each extension reaction to give a final volume of 20 μl Incubation at 37° C. was carried out for 5, 15, and 30 mins.

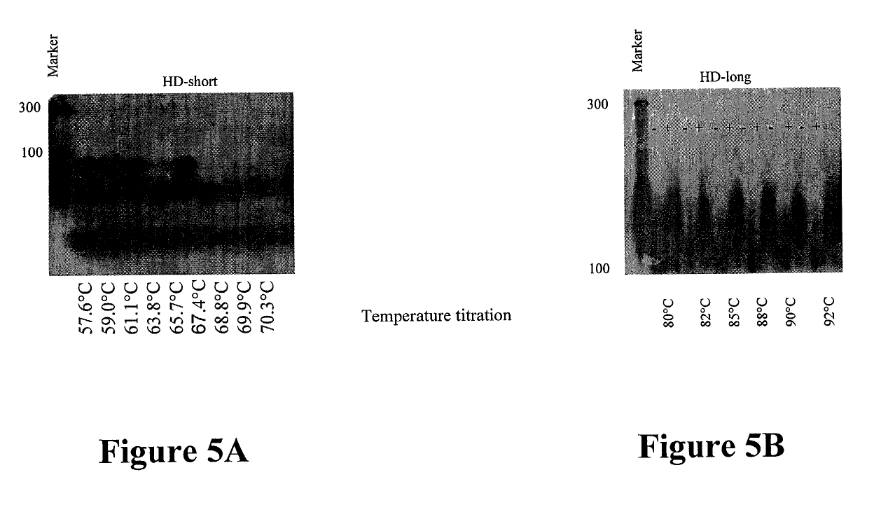

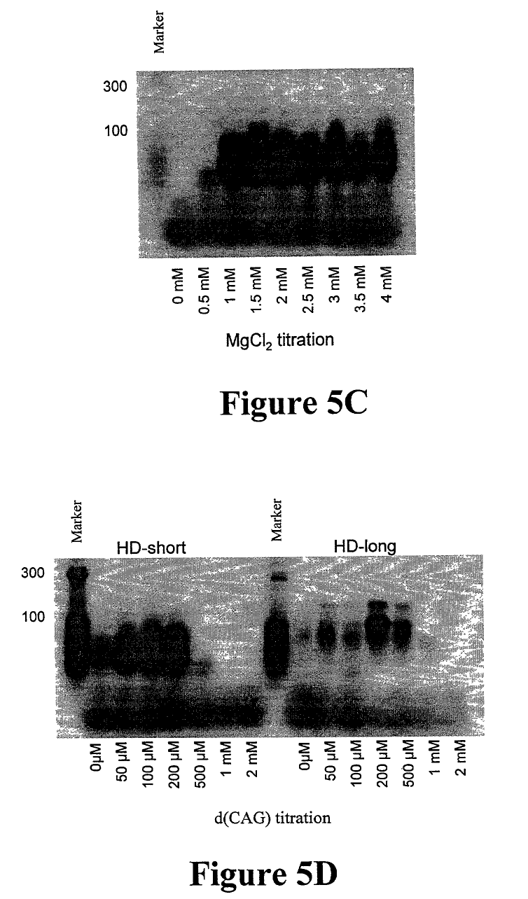

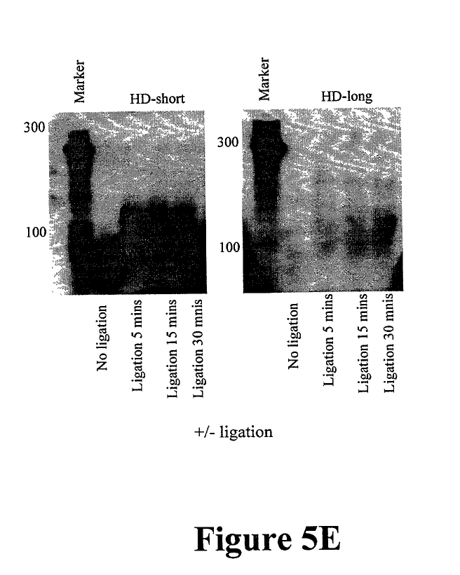

A 2.5% w/v alkaline agarose gel was prepared in alkaline running buffer in order to separate single stranded extension product components (alkaline running buffer consisted of 50 MM NaOH and 1 mM EDTA pH 8.0). The entire 10 μl extension reactions were subjected to electrophoresis at 40V for 5.5 hours (FIG. 5[A],[C],[D],[ ]) or 25V for 14 hours (FIG. 5[B]). The gel was Southern blotted overnight onto Hybond N+(Amersham) using 0.4 M NaOH. The blot was briefly rinsed in 2×SSC and blocked for 30 minutes in 1% blocking reagent (Roche) in buffer 1 (0.1 M Maleic acid, 0.15 M NaCl pH 7.5). The blot was subsequently incubated for 30 minutes in a streptavidin-alkaline phophatase conjugate (Roche) diluted 1:20,000 in 1% blocking reagent in order to detect biotinylated products. Following two, 15 minute washes in 0.3% v/v Tween 20 in buffer 1, the blot was equilibrated in buffer 3 (0.1 M Tris pH 9.5, 0.1 M NaCI) and bands visualized by adding diluted CDP-Star Boehringer-Mannheim) (1:100 in buffer 3) and X-ray exposure.

Single-stranded AFLP DNA markers (30-330 bases) (Life Technologies) were biotinylated using the Biotin-Chem-Link kit from Roche.

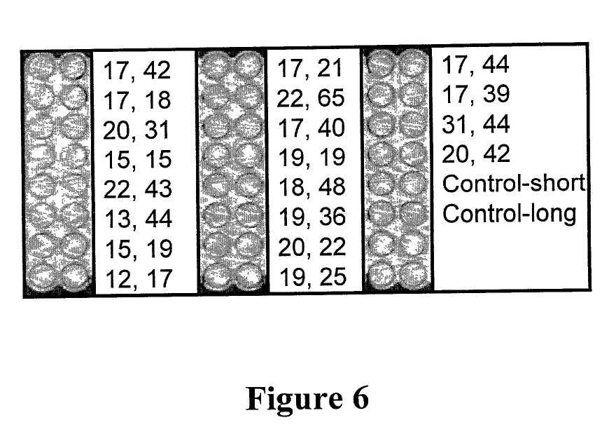

Twenty samples, representing both normal and diseased individuals were subjected to the optimized reaction conditions (FIG. 6) and processed onto streptavidin-coated microtitre plates as described in FIG. 7.

A band was observed of the expected size of 84 bases at ligation temperatures 57.6° C.-63.7° C. for the HD-short template with the short upstream (“us”) oligonucleotide (FIG. 5[A]). Another lower molecular band was also evident of unknown origin. An optimal ligation temperature of 65° C. was chosen for use with the “us” oligonucleotide reactions.

The temperature titration from 55-70° C. for the “us27” oligonucleotide was devoid of the expected 216 base extension product. A titration using higher temperatures was successful in producing a smear of products in the region of the expected size (FIG. 5[B]). The titration was performed with (+) and without (−) Taq Polymerase in order to better visualize the size growth of the 105-mer oligonucleotide (us27) to that of the 216 bases of the extended product.

The 84 base product was observed in reactions with 1.0 to 4.0 mM MgCl₂ (FIG. 5[C]). To maintain minimum influence of this component in the ligation step, a MgCl₂ concentration of 1 mM was selected.

Extension products were visualized over the range 50-200 μM. Interestingly, no products were evident above 200 μM, suggesting some type of inhibitory effect FIG. 5[D]). A noticeably higher product yield for the shorter specific extended product was evident in comparison to the amount of product generated from the longer template with the “us” oligonucleotide.

An increase in product length was evident for both the short (84 to 105 bases) and long (216 to 237 bases) extended products (FIG. 5[E]). However, as previously observed, a significantly lower yield was evident for the HD-long extended and ligated products.

For DNA-based diagnostics to be routinely used in a clinical setting, it must be adapted for large scale screening, with equal or greater accuracy than existing methods and reduced expense. Current diagnosis of Huntington's disease relies heavily on the use of gel electrophoresis, a process that has proved difficult to automate or miniaturize. The present invention provides the LASA methodology to preferentially anneal to its unique position on the template strand. The “us27” oligonucleotide is restricted to only hybridize to (CAG)ₙ regions with 27 or more repeats; the defined lower limit for diseased status. The ligation to a common downstream oligonucleotide, flanking the 3′ end of the (CAG)ₙ repetitive region, is facilitated by a prior extension reaction from the upstream oligonucleotide spanning the (CAG)ₙ region.

The development of LASA provides a unique means of detecting disease-related phenotypes associated with the trinucleotide repeat disorders. The ability of LASA to differentiate between alleles is dependent upon (i) the correct hybridization and extension of one of the upstream oligonucleotides and, (ii) the correct ligation of the correctly extended upstream oligonucleotide. To investigate the total reaction mechanism, it was appropriate to monitor the individual reaction steps. Moreover, it was important to minimise potential inhibitory effects of the extension reaction components upon the ligation reaction. An important requirement for the extension reaction is enzyme fidelity and the absence of 5′-3′ exonuclease activity which would have otherwise displaced the downstream oligonucleotide. AmpliTaq Stoffel fragment from Applied Biosystems satisfied these requirements.

The inventors determined by titration that a concentration of 200 μM d(CAG) optimized the assay. Furthermore, the extension reaction was successful over a range of MgCl₂ concentrations, of which 1 mM was chosen such that it would have minimal effect upon the ligation step. Increasing the reaction temperature provided fidelity and specificity. Two separate temperatures were deemed optimal for the different upstream oligonucleotides, being close to their melting temperature at which complete annealing could be assured. Thus, the strict optimized conditions, particularly the extension temperature, were selected to promote specific binding and extension of the upstream oligonucleotides. Artefactual fragments in the extension reactions shorter than the expected products were commonly detected. However, upon ligation, these did not appear to contribute to the overall absorbance signals.

As expected, the ligation reaction demonstrated an increase in size of the extended product and appeared relatively efficient. No bands of unknown origin were observed. The transfer of these reaction products onto streptavidin-coated microwells was unproblematic. Furthermore, the application of this protocol on 20 human samples clearly demonstrated its usefulness as an alternative diagnostic tool for Huntington's disease detection. The LASA methodology clearly distinguished individuals possessing expanded (CAG)ₙ regions, with 100% accuracy.

The major strength of this technique is its ability to successfully predict Huntington's disease phenotype and totally avoid gel electrophoresis, making it a strong candidate for future use in common laboratories and clinical procedures.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

TABLE 1
Optimized final LASA protocol
Optimized LASA protocol

| | |
|---|---|
| 1. | In a reaction vial, 20 pmoles of the oligo “us”, oligo “ds” |
| | and “spacer” oligonucleotides and 0.2 pmoles of unbiotinylated com- |
| | petitive oligonucleotides are added, with 1 μl of PCR product and 4 |
| | units of Ampligase, a thermostable ligase (Epicentre Technologies) |
| | and appropriate buffer in a final reaction volume of 20 μl. |
| 2. | The reaction mix has an initial denaturation at 94° C. for 5 mins, with |
| | a subsequent 99 cycles of ligation and denaturation steps, i.e. 65° C. |
| | for 60 sec and 94° C. for 10 secs for 99 cycles. (A greater number |
| | of cycles are possible to further increase fluorescence incorporation, |
| | if required.) |
| 3. | 60 μl of a stop buffer (20 × SSC containing 30 mM EDTA) is added |
| | and each reaction mix is added to a streptavidin-coated microtitre |
| | well (NEN-Dupont). Binding is allowed for 30 mins at room temper- |
| | ature. |
| 4. | Two 0.1 M NaOH washes is followed by six washes in PBS/0.1% v/v |
| | Tween 20. |
| 5. | An anti-fluorescein-alkaline phosphatase conjugate (Boehringer- |
| | Mannheim) is diluted 1:1000 in 1% skim milk in 500 mM Tris pH |
| | 7.5/150 mM NaCl. |
| 6. | After a 30 min incubation at room temperature, the wells are washed |
| | six times with PBS/0.1% v/v Tween 20 and three times with PBS. |
| 7. | 100 μl of a PnPP substrate (Sigma) is added and colour is left to |
| | develop. An absorbance reading at 405 nm at 15-120 mins is taken. |

TABLE 2
LASA oligonucleotides for MM211 microsatellite
detection

| Oligo- | | |
|---|---|---|
| nucleotides | Sequence | |
| ATCC-us | 5′-Biotinylated- | (SEQ ID NO:7) |
| | GATTCTGTGATTCTACAACC | |
| ATCC-ds | 5′-Phosphorylated- | (SEQ ID NO:8) |
| | ACCCACAGACCTCTTCCCAC- | |
| | 3′ Fluorescein | |
| ATCC-4 | 5′-Phosphorylated- | (SEQ ID NO:9) |
| | ATCCATCCATCCATCC | |
| ATCC-9 | 5′-Phosphorylated- | (SEQ ID NO:10) |
| | ATCCATCCATCCATCCATCCAT | |
| | CCATCCATCCATCC | |
| ATCC-10 | 5′-Phosphorylated | (SEQ ID NO:11) |
| | ATCCATCCATCCATCCATCCAT | |
| | CCATCCATCCATCCATCC | |
| ATCC-11 | 5′-Phosphorylated | (SEQ ID NO:12) |
| | ATCCATCCATCCATCCATCCAT | |
| | CCATCCATCCATCCATCCATCC | |
| ATCC-12 | 5′-Phosphorylated- | (SEQ ID NO:13) |
| | ATCCATCCATCCATCCATCCAT | |
| | CCATCCATCCATCCATCCATCC | |
| | TCC | |
| Competitive- | 5′-GATTCTGTGATTCTACAAC | (SEQ ID NO:14) |
| us + 9 | CATCCATCCATCCATCCATCCA | |
| | TCCATCCAT | |
| Competitive- | 5′-GATTCTGTGATTCTACAAC | (SEQ ID NO:15) |
| us + 10 | CATCCATCCATCCATCCATCCAT | |
| | CCATCCATCCATCCATCCATCCA | |
| | TCC | |
| Competitive- | 5′-GATTCTGTGATTCTACAACC | (SEQ ID NO:16) |
| us + 11 | ATCCATCCATCCATCCATCCAT | |
| | CCATCCATCCATCCATCCATCC | |
| Competitive- | 5′-GATTCTGTGATTCTACAAC | (SEQ ID NO:17) |
| us + 12 | CATCCATCCATCCATCCATCCA | |
| | TCCATCCATCCATCCATCCATC | |
| | CATCC | |

TABLE 3
Comparison of LASA reactions with and without cycling of denaturation
and ligation

Absorbance 405 nm

| SPACER OLIGO | Non-cycling | Cycling |
|---|---|---|
| No template control | 0.045 | 0.094 |
| ATCC-4 | 0.348 | 0.133 |
| ATCC-9 | 0.557 | 0.279 |
| ATCC-10 | 0.421 | 1.055 |
| ATCC-11 | 0.320 | 0.277 |
| ATCC-12 | 0.187 | 0.378 |

TABLE 4
Titration of template concentration in the LASA assay

| | | | 50 | 100 | 250 | 500 | | | 10 | “25” | 50 | 100 |
|---|---|---|---|---|---|---|---|---|---|---|---|---|
| | 0 | 5 amol | amol | amol | amol | amol | 1 pmol | 5 pmol | pmol | pmol | pmol | pmol |
| ATCC-9 | 0.071 | 0.073 | 0.103 | 0.176 | 0.501 | 0.727 | 0.891 | 1.241 | 1.103 | 1.298 | 0.165 | 0.132 |
| ATCC-10 | 0.114 | 0.251 | 0.479 | 0.710 | 1.772 | 2.005 | 2.266 | 2.910 | 1.318 | 2.445 | 2.697 | 0.627 |
| ATCC-11 | 0.041 | 0.044 | 0.057 | 0.057 | 0.072 | 0.247 | 0.189 | 0.166 | 0.265 | 0.107 | 0.094 | 0.191 |
| ATCC-12 | 0.046 | 0.048 | 0.058 | 0.058 | 0.062 | 0.285 | 0.138 | 0.231 | 0.098 | 0.196 | 0.051 | 0.144 |

Equivalent to 5 μl of PCR product

TABLE 5
LASA assay results performed on 12 bird samples

| | 46766 | 48385 | 28102 | D782 | 20970 | 46765 | 48937 | 48903 | D783 | MM100 | MM104 | 28103 |
|---|---|---|---|---|---|---|---|---|---|---|---|---|
| spacer 9 | 0.438 | 0.089 | 0.057 | 1.109 | 0.088 | 0.149 | 0.106 | 0.056 | 0.180 | 1.985 | 0.125 | 1.169 |
| spacer 10 | 1.378 | 1.113 | 1.293 | 0.200 | 0.131 | 1.319 | 1.460 | 0.078 | 1.965 | 3.264 | 1.146 | 1.401 |
| spacer 11 | 0.209 | 0.217 | 0.263 | 0.146 | 1.124 | 0.588 | 0.273 | 1.088 | 0.124 | 0.135 | 0.218 | 0.287 |
| spacer 13 | 0.177 | 0.103 | 0.142 | 0.096 | 0.051 | 0.146 | 0.111 | 0.172 | 0.381 | 0.273 | 0.083 | 0.566 |
| LASA | 10,10 | 10,10 | 10,10 | 9,9 | 11,11 | 10,10 | 10,10 | 11,11 | 10,10 | 9,10 | 10,10 | 9,10 |
| Genotype | ? | | | | | | | | | | | |
| Genotype | 9.10 | 10,10 | 10,10 | 9,9 | 9,11 | 10,10 | 10,10 | 10,10 | 10,10 | 9,10 | 10,10 | 9,10 |
| (Seq Gel) | | | | | | | | | | | | |
| | ↑ | | | | ↑ | | | | | | | |

TABLE 6
Titration of competing oligonucleotides

| Spacer | | | | | | | | | | |
|---|---|---|---|---|---|---|---|---|---|---|
| oligo | 0 pmol | 0.1 pmol | 0.2 pmol | 0.5 pmol | 1.0 pmol | 2.0 pmol | 5 pmol | 10 pmol | 20 pmol | 50 pmol |
| ATCC-9 | 1.607 | 0.491 | 0.147 | 0.076 | 0.110 | 0.078 | 0.054 | 0.051 | 0.054 | 0.051 |
| ATCC-10 | 3.863 | 1.673 | 1.002 | 0.0158 | 0.197 | 0.164 | 0.062 | 0.058 | 0.054 | 0.051 |
| ATCC-11 | 0.161 | 0.233 | 0.200 | 0.179 | 0.079 | 0.065 | 0.076 | 0.056 | 0.053 | 0.049 |
| ATCC-12 | 0.153 | 0.087 | 0.135 | 0.077 | 0.74 | 0.77 | 0.068 | 0.052 | 0.056 | 0.051 |
| | ↑ | | ↑ | | | | | | | |
| | without | | Optimal | | | | | | | |
| | competitive | | competitive | | | | | | | |
| | oligonucleotides | | oligonucleo- | | | | | | | |
| | | | tide concentra- | | | | | | | |
| | | | tion | | | | | | | |

TABLE 7
LASA varying both template and competitive oligonucleotide
concentrations

| | | | | 0.2 pmoles | | | 0.4 pmoles | | |
|---|---|---|---|---|---|---|---|---|---|
| | No | | | competitive | | | competitive | | |
| | competitive oligonucleotides | | | oligonucleotides | | | oligonucleotides | | |
| | 1/10 | 1 | 5 | 1/10 | 1 | 5 | 1/10 | 1 | 5 |
| ATCC- | 0.450 | 2.007 | 1.179 | 0.078 | .0233 | 0.338 | 0.057 | 0.100 | .0161 |
| 9 | | | | | | | | | |
| ATCC- | 2.189 | 3.749 | 3.088 | 1.620 | 3.057 | 2.859 | 0.674 | 0.924 | 1.452 |
| 10 | | | | | | | | | |
| ATCC- | .0219 | 0.380 | 1.130 | 0.043 | 0.067 | 0.080 | 0.140 | 0.190 | 0.645 |
| 11 | | | | | | | | | |
| ATCC- | 0.169 | .0325 | 1.062 | 0.075 | 0.111 | 0.211 | 0.056 | 0.079 | 0.113 |
| 12 | | | | | | | | | |

TABLE 8
Ligation temperature titration

| | Absorbance 405 nm | | | | | | |
|---|---|---|---|---|---|---|---|
| | 55.6° | 56.8° | 58.8° | 61.6° | 65.0° | 69.0° | 72.5° |
| Spacer | C. | C. | C. | C. | C. | C. | C. |
| ATCC-9 | 1.638 | 1.297 | 1.968 | 1.001 | 0.120 | 0.072 | 0.049 |
| ATCC-10 | 2.984 | 3.333 | 2.488 | 2.212 | 1.078 | 0.525 | 0.047 |
| ATCC-11 | 2.061 | 1.548 | 0.832 | 0.274 | 0.72 | 0.047 | 0.045 |
| ATCC-12 | 0.281 | 0.481 | 0.131 | 0.119 | 0.053 | 0.051 | 0.42 |

[image] TABLE 10
LASA assay results performed on 12 bird samples (arrows indicate discrepant results)

| | Bird Sample | | | | | | | | | | | |
|---|---|---|---|---|---|---|---|---|---|---|---|---|
| | 46766 | 48385 | 28102 | D782 | 20970 | 46765 | 48937 | 48903 | D783 | MM1100 | MM104 | 28103 |
| spacer 9 | 1.292 | 0.089 | 0.057 | 1.109 | 0.823 | 0.149 | 0.106 | 0.857 | 0.180 | 1.985 | 0.125 | 1.169 |
| spacer 10 | 1.763 | 1.113 | 1.293 | 0.200 | 1.174 | 1.319 | 1.460 | 1.774 | 1.965 | 3.265 | 1.146 | 1.401 |
| spacer 11 | 0.070 | 0.217 | 0.263 | 0.146 | 1.022 | 0.388 | 0.273 | 0.144 | 0.124 | 0.135 | 0.218 | 0.287 |
| spacer 12 | 0.061 | 0.103 | 0.142 | 0.096 | 0.109 | 0.146 | 0.111 | 0.105 | 0.381 | 0.273 | 0.083 | 0.366 |
| LASA | 9,10 | 10,10 | 10,10 | 9,9 | 9,10,11 | 10,10 | 10,10 | 9,10 | 10,10 | 9,10 | 10,10 | 9,10 |
| Genotype | | | | | | | | | | | | |
| Genotype | 9,10 | 10,10 | 10,10 | 9,9 | 9,11 | 10,10 | 10,10 | 10,10 | 10,10 | 9,10 | 10,10 | 9,10 |
| (Seq Gel) | | | | | | | | | | | | |

TABLE 11
LASA oligonucleotides designed for the human D1S191 microsatellite length measurement

| OLIGONUCLEOTIDE | SEQUENCE | |
|---|---|---|
| PCR PRIMERS | | |
| D15191-UpStream | 5′Biotin-GCA TTTGCTTACAAATATCCTA | (SEQ ID NO:18) |
| D15191-DownSream | 5′phoshate-CTTTAAAGGAGGACTGGCTTGTAT-3′ Fluorescein | (SEQ ID NO:19) |
| SPACER | | |
| OLINUCLEOTIDES: | | |
| CA-1 | 5′Phosphate-CA | (SEQ ID NO:20) |
| CA-17 | 5′Phosphate-CACACACACACACACACACACACACACACACA | (SEQ ID NO:21) |
| CA-18 | 5′Phosphate-CACACACACACACACACACACACACACACACACA | (SEQ ID NO:22) |
| CA-19 | 5′Phosphate-CACACACACACACACACACACACACACACACACACA | (SEQ ID NO:23) |
| CA-20 | 5′Phosphate-CACACACACACACACACACACACACACACACACACACA | (SEQ ID NO:24) |
| CA-21 | 5′Phosphate-CACACACACACACACACACACACACACACACACACACACA | (SEQ ID NO:25) |
| CA-22 | 5′Phosphate-CACACACACACACACACACACACACACACACACACACACACA | (SEQ ID NO:26) |
| CA-23 | 5′Phosphate-CACACACACACACACACACACACACACACACACACACACACACA | (SEQ ID NO:27) |
| CA-24 | 5′Phosphate-CACACACACACAcACACACACACACACACACACACACACACACACA | (SEQ ID NO:28) |
| CA-25 | 5′Phosphate-CACACACACACACACACACACACACACACACACACACACACACACACA | (SEQ ID NO:29) |
| COMPETITVE | | |
| OLIGONUCLEOTIDES: | | |
| US + CA₁₇ | 5′Phosphate-ATTTGCTTACAAATATCCTACACACACACACACACACACACACACACACACACA | (SEQ ID NO:30) |
| US + CA₁₈ | 5′Phosphate-ATTTGCTTACAAATATCCTACACACACACACACACACACACACACACACACACACA | (SEQ ID NO:31) |
| US + CA₁₉ | 5′Phosphate-ATTTGCTTACAAATATCCTACACACACACACACACACACACACACACACACACACACA | (SEQ ID NO:32) |
| US + CA₂₀ | 5′Phosphate-ATTTGCTTACAAATATCCTACACACACACACACACACACACACACACACACACACACA | (SEQ ID NO:33) |
| CA | | |
| US + CA₂₁ | 5′Phosphate-ATTTGCTTACAAATATCCTACACACACACACACACACACACACACACACACACACACA | (SEQ ID NO:34) |
| CACA | | |
| US + CA₂₂ | 5′Phosphate-ATTTGCTTACAAATATCCTACACACACACACACACACACACACACACACACACACACA | (SEQ ID NO:35) |
| CACACA | | |
| US + CA₂₃ | 5′Phosphate-ATTTGCTTACAAATATCCTACACACACACACACACACACACACACACACACACACACA | (SEQ ID NO:36) |
| CACACACA | | |
| US + CA₂₄ | 5′Phosphate-ATTTGCTTACAAATATCCTACACACACACACACACACACACACACACACACACACACA | (SEQ ID NO:37) |
| CACACACACA | | |
| US + CA₂₅ | 5′Phosphate-ATTTGCTTACAAATATCCTACACACACACACACACACACACACACACACACACACACA | (SEQ ID NO:38) |

CACACACACACA

TABLE 12
Both non-competitive and competitive LASA reactions were conducted over a temperature range of 55-70° C. PCR
product from a plasmid containing (CA)₂₀ repeats was used as a standard in all optimizations. Little discrimination
between the correctly matched and incorrectly matched spacer oligonucleotides was evident in the non-competitive
LASA. The inclusion of 0.2 pmoles of competitive oligonucleotides made only slight improvements in the
differentiation between the correct and incorrect spacer incorporation.

| | NON-COMPETITIVE | | | | | | | | | | | |
|---|---|---|---|---|---|---|---|---|---|---|---|---|
| SPACER | 55.4° C. | 55.8° C. | 56.4° C. | 57.7° C. | 59.4° C. | 61.4° C. | 63.3° C. | 65.3° C. | 67.6° C. | 69.0° C. | 69.7° C. | 70.2° C. |
| CA₁₈ | 2.590 | 2.544 | 2.457 | 2.338 | 2.304 | 1.767 | 1.805 | 1.470 | 0.688 | 0.354 | 0.265 | 0.196 |
| CA₁₉ | 2.834 | 2.713 | 2.725 | 2.142 | 2.414 | 1.503 | 2.158 | 1.452 | 1.184 | 0.633 | 0.524 | 0.141 |
| CA₂₀ | 2.959 | 2.817 | 3.167 | 9.603 | 3.116 | 2.049 | 2.312 | 1.725 | 1.625 | 0.826 | 0.699 | 0.128 |
| CA₂₁ | 2.697 | 2.551 | 2.734 | 2.645 | 2.498 | 1.849 | 1.739 | 1.149 | 0.751 | 0.441 | 0.297 | 0.281 |
| CA₂₂ | 1.392 | 1.639 | 2.182 | 1.998 | 1.538 | 1.666 | 1.091 | 0.518 | 0.273 | 0.222 | 0.150 | 0.141 |
| | COMPETITIVE | | | | | | | | | | | |
| SPACER | 55.1° C. | 55.3° C. | 56.4° C. | 57.6° C. | 59.2° C. | 61.1° C. | 63.8° C. | 65.7° C. | 67.4° C. | 68.8° C. | 69.9° C. | 70.3° C. |
| CA₁₈ | 0.211 | 0.244 | 0.215 | 0.196 | 0.170 | 0.166 | 0.1525 | 0.082 | 0.083 | 0.058 | 0.056 | 0.050 |
| CA₁₉ | 0.345 | 0.322 | 0.298 | 0.333 | 0.229 | 0.217 | 0.150 | .0130 | 0.082 | 0.055 | 0.061 | 0.059 |
| CA₂₀ | 0.780 | 0.705 | 0.628 | 0.606 | 0.314 | 0.148 | 0.342 | 0.409 | 0.247 | 0.161 | 0.107 | 0.068 |
| CA₂₁ | 0.706 | 0.672 | 0.643 | 0.571 | 0.565 | 0.496 | 0.388 | 0.223 | 0.042 | 0.078 | 0.075 | 0.060 |
| CA₂₂ | 0.520 | 0.639 | 0.474 | 0.476 | 0.588 | 0.344 | 0.258 | 0.113 | 0.039 | 0.054 | 0.052 | 0.052 |

**Shading denotes expected positive result

TABLE 13
Template denaturation prior to the addition of LASA reagents in the
presence of NaCl, over the range 0 to 0.9 M demonstrated a reduction in
the signal to noise ratio to less than 22% at 0.18 M NaCl concentration.
(For simplicity, only (CA)₁₉ to (CA)₂₁ were tested).

| SPAC- | SALT CONCENTRATION DURING TEMPLATE DENATURATION | | | | | | |
|---|---|---|---|---|---|---|---|
| ER | 0 M | 0.09 M | 0.18 M | 0.27 M | 0.36 M | 0.45 M | 0.9 M |
| CA₁₉ | 3.042 | 2.214 | 0.262 | 0.132 | 0.120 | 0.139 | 0.140 |
| CA₂₀ | 3.301 | 2.857 | 1.248 | 0.280 | 0.142 | 0.146 | 0.151 |
| CA₂₁ | 1.874 | 0.775 | 0.183 | 0.143 | 0.141 | 0.124 | 0.126 |

↑
Optimal NaCl concentration

TABLE 14
The effect of temperature using prior denaturation of the template in the presence of 0.18 M NaCl was examined.
Optimal signal to noise ratios were encountered between 65.7° C. to 67.4° C. Thus 66° C. was selected for all future
experiments.

| SPACER | 55.1° C. | 55.5° C. | 56.4° C. | 57.6° C. | 59.2° C. | 61.1° C. | 63.8° C. | 65.7° C. | 67.4° C. | 68.8° C. | 69.9° C. | 70.3° C. |
|---|---|---|---|---|---|---|---|---|---|---|---|---|
| CA₁₉ | 0.988 | 1.253 | 1.900 | 1.690 | 1.400 | 1.486 | 0.904 | 0.599 | 0.351 | 0.145 | 0.133 | 0.131 |
| CA₂₀ | 3.017 | 3.226 | 2.720 | 2.958 | 2.194 | 2.609 | 2.859 | 2.517 | 1.286 | 0.418 | 0.354 | 0.272 |
| CA₂₁ | 1.447 | 1.710 | 1.698 | 1.310 | 1.956 | 1.431 | 1.405 | 0.654 | 0.297 | 0.156 | 0.136 | 0.132 |

TABLE 15
Optimized protocol for detecting Huntington's disease
Optimized LASA protocol

| | |
|---|---|
| 1. | 5 μl of PCR products were added to one of two reaction tubes con- |
| | taining 1 mM MgCl₂, 200 μM d(CAG) mix, 2X extension buffer |
| | (supplied by manufacturer), 5 pmoles of “ds” oligonucleotide, 0.5 |
| | units AmpliTaq Stoffel fragment polymerase (Applied Biosystems) |
| | and 5 pmoles of either “ds” of “us27” oligonucleotide. |
| 2. | Extension reaction for “us”: 94° C./5 mins (94° C. for 10 secs, |
| | 65° C. for 20 secs) X99. Extension reaction for “us27”: |
| | 94° C./5 mins (94° C. for 10 secs, 85° C. for 10 secs) X99. |
| 3. | 1 unit of T4 DNA ligase, 2 μl ligase buffer and water to a final vol- |
| | ume of 20 μl are added to each extension reaction vial. Incubation is |
| | conducted at 37° C. for 1 hour. |
| 4. | 80 μl of inactivation/binding buffer (20X SSC containing 30 mM |
| | EDTA) was added and reactions were bound onto streptavidin plates |
| | for 30 mins. |
| 5. | The plate was washed twice in 0.2 M NaOH, six times in PBS/0.1% |
| | v/v Tween-20. |
| 6. | A further 30 mins incubation was conducted with anti-fluorescein-al- |
| | kaline phosphatase conjugate (Roche) diluted 1:1,000 in 1% w/v |
| | Skim milk in 100 mM Tris pH 7.5/150 mM NaCl. |
| 7. | The plate was again washed six times in PBS/0.1% v/v Tween-20, |
| | three times in PBS only. |
| 8. | 100 μl of a PnPP substrate solution (Sigma) was added to each well |
| | and an overnight absorbance reading was taken at 405 nm. |

### BIBLIOGRAPHY

Amos et al (1993) Nature Genetics 13:390-391

Beaucage et al. (1981) Tetrahedron Letters 22:1859-1862

Brown et al. (1979) Methods Enzymol 68:109

Campuzano et al. (1996) Science 271:1423-1427

Gill et al (1985) Nature 318:577-579

Gyapay et al. (1994) Nature Genetics 7:246-339

Heame et al (1992) Trends in Genetics 8:288-294

Huntington's Disease Collaboration Research Group (1993) Cell 72:971-983

Jeffreys et al. (1989) Nucleic Acids Research 16:10953-10971

Klinlicht, M. and Tautz, D. (1992) Molecular Ecology 1:133-134

Knowles et al. (1992) American Journal of Human Genetics 51:905-909

Kristensen, V. N. and Dale, A. B. (1997) Biotechniques 23:634-636

Lins et al (1996) Biotechniques 20:882-888

Longmire et al (1993) The Auk 110:378-381

MacDonald, D. B. and Potts, W. K. (1994) Science 266:1030-1032

Mathies, R. A (1995) Methods in Enzymology 246:377-389

Primmer et al. (1995) MolecularEcology 4493-498

Rousseau et al. (1991) Journal of Medical Genetics 28:830-836

Roy et al. (1994) Molecular Biology and Evolution 11:553-570

Strassman et al. (1996) Strategies for finding a nd using highly polymorphic DNA microsatellite loci for studies of genetic relatedness and pedigrees, in Molecular Zoology: Advances, Strategies and Protocols, J. D. Ferrarris, S. R. Palumbi (eds), Wiley-Liss, New York

Sutherland et al. (1993) Medical Journal of Australia 158;482-485

Taranenko et al. (1999) Biomolecular engineering 14:25-31

Tautz, R. (1989) Nucleic Acids Research 17:6463-6471

Taylor et al. (1994) Molecular Ecology 3:277-290

Tegelstrom, H. ((1986) Electrophoresis 7:22&229

Tereba et al. (1998) Biotechniques 25(5):892-897

Vuillaume et al. (1998) Diagnostic Molecular Pathology 7(3):174-179

Zirvi et al. (1999a) Nucleic Acids Research 27(24):e40

Zirvi et al. (1999b) Nucleic Acids Research 27(24):e41

1. A method for identifying or otherwise detecting a nucleotide repeat region, characterized by a particular length, in a nucleic acid molecule, said method comprising annealing to a single stranded template from said nucleic acid molecule a set of oligonucleotides, said set comprising at least two flanking oligonucleotides which are capable of annealing to nucleotide sequences on the template nucleic acid molecule flanking the nucleotide repeat region and at least one spacer oligonucleotide capable of annealing to a nucleotide sequence defining all or part of the nucleotide repeat region and wherein one of said flanking oligonucleotides is labelled with a capturable moiety and the other of said flanking oligonucleotide is labelled with a detectable moiety and subjecting the annealed molecules to a ligation reaction sufficient to permit ligation of two or more oligonucleotides if ligatably adjacent to each other and then subjecting the ligation product to conditions to facilitate attachment of the capturable moiety to a binding partner immobilized to a solid support and then subjecting the immobilized molecule to denaturing conditions to separate the template nucleic acid molecule from the annealed, potentially ligated oligonucleotides and then screening for said detectable moiety on the solid support and wherein the presence of said detectable moiety is indicative that said spacer oligonucleotide is ligated to the flanking oligonucleotides, the length of said spacer oligonucleotide thereby corresponding to the length of the nucleotide repeat region.

2. A method of identifying or otherwise detecting a nucleotide repeat region characterized by a particular length in a nucleic acid molecule, said method comprising annealing to a single stranded template form said nucleic acid molecule at least two flanking oligonucleotides which flank the putative nucleotide repeat region to be identified and, in a multiplicity of separate reactions, a spacer oligonucleotide of a defined length in each separate reaction which spacer oligonucleotide anneals to all or part of the nucleotide sequence between said flanking oligonucleotides wherein one of said flanking oligonucleotides is labelled with a capturable moiety and the other of said flanking oligonucleotide is labelled with a detectable moiety and subjecting said annealed molecules to ligation reactions and attachment conditions such that the oligonucleotide comprising a terminal capturable moiety anchors the annealed, potentially ligated nucleic acid molecule to a solid support; subjecting said anchored nucleic acid molecule to denaturing means such that the template nucleic strand of the nucleic acid molecule separates from the annealed oligonucleotides and then screening for said detectable moiety on a flanking oligonucleotide wherein the presence of a detectable signal is indicative that the three oligonucleotides are in tandem ligatable arrangement wherein the spacer oligonucleotide in the reaction giving the signal corresponds to the length of the nucleotide repeat region.

3. A method of identifying or otherwise detecting a nucleotide repeat region characterized by a particular length in a nucleic acid molecule, said method comprising annealing to a single stranded template form said nucleic acid molecule at least two flanking oligonucleotides which flank the putative nucleotide repeat region to be identified and, in a multiplicity of separate reactions, a spacer oligonucleotide of a defined length in each separate reaction which spacer oligonucleotide anneals to all or part of the nucleotide sequence between said flanking oligonucleotides wherein one of said flanking oligonucleotides is labelled with a capturable moiety and the other of said flanking oligonucleotide is labelled with a detectable moiety and subjecting said annealed molecules to ligation reactions and attachment conditions such that the oligonucleotide comprising a terminal capturable moiety anchors the annealed, potentially ligated nucleic acid molecule to a solid support; subjecting said anchored nucleic acid molecule to denaturing means such that the template nucleic strand of the nucleic acid molecule separates from the annealed oligonucleotides and then screening for said detectable moiety on a flanking oligonucleotide wherein the presence of a detectable signal is indicative that the three oligonucleotides are in tandem ligatable arrangement wherein the spacer oligonucleotide in the reaction giving the signal corresponds to the length of the nucleotide repeat region.

4. A method according to claim 1 or 2 or 3 wherein the spacer oligonucleotide region is from about 2 to about 400 nucleotides in length.

5. A method according to claim 4 wherein the spacer oligonucleotide region is from about 2 to about 200 nucleotides in length.

6. A method according to claim 5 wherein the spacer nucleotide region is from about 2 to about 120 nucleotides in length.

7. A method according to claim 1 or 2 or 3 wherein the template nucleic acid molecule is from a nucleic acid molecule which has been subject to amplification.

8. A method according to claim 7 wherein the amplification reaction is PCR, rolling circle amplification or Qβ replicase-based amplification.

9. A method according to claim 8 wherein the amplification reaction is PCR.

10. A method according to claim 1 or 2 or 3 wherein the nucleic acid molecule is DNA.

11. A method according to claim 10 wherein the nucleic acid molecule is cDNA.

12. A method according to claim 1 or 2 or 3 wherein the nucleotide repeat region is characteristic of a new degenerative disease.

13. A method according to claim 12 wherein the neurodegenerative disease is fragile X syndrome, Huntington's disease or muscular dystrophy.

14. A method according to claim 13 wherein the neurodegenerative disease is Huntington's disease.

15. A method according to claim 1 or 2 or 3 wherein the solid support is glass or a polymer.

16. A method according to claim 15 wherein the polymer is cellulose and its derivatives, ceramic material, nitrocellulose, polyacrylamide, nylon, polystyrene and it derivatives, polyvinyl chloride or polypropylene.

17. A method for determining the length of a nucleotide repeat region such as in the form of a microsatellite in a target nucleic acid molecule, said method comprising the steps of:
(i) obtaining a sample of said target nucleic acid molecule;
(ii) optionally amplifying the repeat region on said target nucleic acid molecule;
(iii) subjecting the target nucleic acid molecule to denaturing conditions to yield a single stranded template carrying the repeat region;
(iv) annealing to said template three oligonucleotides separating, sequentially or simultaneously wherein two oligonucleotides are flanking oligonucleotides which are capable of annealing to the template at positions flanking the nucleotide repeat region and the third oligonucleotide is of a defined length and complementary to the nucleotide repeat region and wherein one of said flanking oligonucleotides is labelled at one end with a capturable moiety and the other flanking oligonucleotide is labelled at an end opposite to the first mentioned flanking oligonucleotide with a detectable moiety;
(v) subjecting the annealed oligonucleotides-template complex to ligation conditions such that the flanking oligonucleotides ligate to the spacer oligonucleotide if the spacer oligonucleotide is ligatably adjacent the flanking oligonucleotides;
(vi) subjecting the ligation product to anchoring conditions to capture the flanking oligonucleotide carrying the capturable moiety to a solid support;
(vii) subjecting the captured ligation product to denaturing means to release the template; and
(viii) screening for an identifiable signal wherein the presence of a signal is indicative of a spacer oligonucleotide corresponding to the length of the nucleotide repeat region.

18. Use of ligase assisted spacer addition (LASA) in the identification of a nucleotide length polymorphism in an animal or human subject.

19. Use according to claim 18 wherein the polymorphism is associated with a neurodegenerative disease.

20. Use according to claim 19 wherein the neurodegenerative disease is fragile X syndrome, Huntington's disease or muscular dystrophy.

21. Use according to claim 20 wherein the neurodegenerative disease is Huntington's disease.

22. A composite nucleotide sequence comprising the structure [x_{I}x_{II} . . . xₙ](●)ₐ8 y_{I}y_{II} . . . yₘ](●)_{b}[z_{I}z_{II} . . . zₒ]
wherein
[x_{I}x_{II} . . . xₙ] and [z_{I}z_{II} . . . zₒ] are oligonucleotides of length n and o, respectively, capable of annealing to two nucleotide sequences flanking a nucleotide repeat region on a nucleic acid molecule;
[y_{I}y_{II} . . . yₘ] is an oligonucleotide of length m and capable of annealing to a nucleotide repeat region between the two flanking nucleotides [x_{I}x_{II} . . . xₙ] and [z_{I}z_{II} . . . zₒ];
(●)ₐ and (●)_{b} represent phosphodiester bonds between adjacent nucleotides wherein ₐ and _{b} may be the same or different and each is 0 or 1 and wherein when ₐ and/or _{b} is 0, the adjacent oligonucleotides are not ligated together;
wherein said composite oligonucleotide is formed by the process comprising annealing x, y and z separately or simultaneously to a singled stranded template nucleic acid molecule comprising a nucleotide repeat region wherein x and z anneal to regions flanking y, subjecting the molecules to ligation to generate (●)ₐ and (●)_{b} wherein ₐ and _{b} are both 1 if y is ligatably adjacent x and z on the template; immobilizing the ligated product to a solid support and subjecting the immobilized product to denaturing conditions to remove the template and then detecting the presence of the composite oligonucleotide wherein the presence of a composite oligonucleotide is indicative that y is ligatable adjacent x and z.

23. A method for discriminating between nucleotide repeat regions characterized by particular lengths in a nucleic acid molecule, said method comprising annealing to a single-stranded template from said nucleic acid molecule a set of oligonucleotides wherein one oligonucleotide anneals upstream of a putative nucleotide repeat region and is of a length which is shorter than the repeat region or is longer than said repeat region and a second oligonucleotide anneals downstream of said repeat region and wherein one of said upstream or downstream oligonucleotides is labelled with a capturable moiety and the other of said upstream of downstream oligonucleotides is labelled with a detectable moiety and then subjecting said upstream oligonucleotide to nucleotide extension conditions whereby if the upstream oligonucleotide is shorter than the repeat region, the extension product becomes ligatably adjacent the downstream oligonucleotide whereas if the upstream oligonucleotide is longer than the repeat region, then ligation is not possible with said downstream oligonucleotides such that upon ligation and immbolization to a solid support, the presence or absence of a detectable signal is indicative of an upstream oligonucleotide of a particular length and thereby a repeat region of a particular length.

24. A method according to claim 23 wherein the method is conducted in duplicate with one or two upstream oligonucleotides wherein one of said oligonucleotides is potentially longer than said repeat region and the other oligonucleotide is potentially shorter than said repeat region and/or both oligonucleotides are potentially shorter than said repeat reigon.

25. A method according to claim 23 or 24 for the detection of a neurodegenerative disease.

26. A method according to claim 25 wherein the neurodegenerative disease is Huntington's disease.

27. A computer program-assisted method for detecting or identifying a nucleotide length polymorphism, said method comprising:
(i) means to perform LASA or a related method; and
(ii) data processing means to record the presence of an identifiable signal and correlating same to the size of a spacer oligonucleotide.
